(19) United States
(12) Patent Application Publication
(10) Pub. No.: US 2021/0393538 A1
(43) Pub. Date: Dec. 23, 2021

(54) SILICA-BASED DRUG NANOCAPSULES

(71) Applicant: University of Georgia Research Foundation, Inc., Athens, GA (US)

(72) Inventors: Jin XIE, Athens, GA (US); Weizhong ZHANG, Athens, GA (US)

(21) Appl. No.: 17/289,384

(22) PCT Filed: Oct. 29, 2019
(86) PCT No.: PCT/US2019/058525

§ 371 Date: Apr. 28, 2021

Related U.S. Application Data
(60) Provisional application No. 62/751,833, filed on Oct. 29, 2018.

(51) Int. Cl. A61K 9/51; A61K 45/06; A61K 9/19; A61K 9/00

(52) U.S. Cl. CPC A61K 9/5115; A61K 45/06; B82Y 5/00; A61K 9/0019; A61K 9/19

(57) ABSTRACT

Disclosed herein are nanoparticles comprising a therapeutic or diagnostic agent positioned within an orthosilicate inner matrix, and a silica outer shell encapsulating the orthosilicate inner matrix. Also disclosed are methods of treating a disease in a subject comprising administering to the subject a biological carrier cell comprising the nanoparticle. Also disclosed are methods of preparing nanoparticles, methods of delivering nanoparticles comprising a therapeutic or diagnostic agent to a target cell, and methods of loading a therapeutic or diagnostic agent into a biological carrier cell.

### CROSS-REFERENCE TO RELATED APPLICATIONS

This application claims benefit of U.S. Provisional Application No. 62/751,833, filed Oct. 29, 2018, incorporated herein by reference in its entirety.

### STATEMENT REGARDING FEDERALLY SPONSORED RESEARCH

This invention was made with government support under Grant No. R01EB022596 awarded by the National Institutes of Health. The Government has certain rights in the invention.

### FIELD

The disclosure generally relates to nanoparticles, particularly silica-based nanoparticles useful for drug delivery.

### BACKGROUND

Exploiting immune cells for drug delivery is an emerging area of research.^{[1-3]} Many types of leukocytes, including macrophages, neutrophils, and dendritic cells, can sense chemokine and cytokine cues and home to inflamed tissues. Macrophages or their predecessor monocytes in particular, can respond to cancer-related cytokines (e.g., CSF-1, VEGF, PDGF, TNF, IL-1, IL-5, etc.) and chemokines (e.g., CCL-5, 7, 8, 12, etc.),^{[4-6]} and navigate to the diseased sites, passing multiple biological barriers along the way. This holds true for central tumor areas, which are often avascular and inaccessible to conventional therapeutics. These unique properties make macrophages a potentially appealing vehicle for cancer drug delivery.

Despite the promises, it remains a challenge to load large quantities of drugs into macrophages. Conventionally, the most common cell loading strategy is to conjugate drug molecules or tether drug-loaded nanoparticles onto the cell plasma membrane.^{[7]} This so-called “backpack” approach has been exploited by others and us to load therapeutics onto stem cells,^{[8-11]} leukocytes,^{[12-15]} red blood cells,^{[16]} and T cells^{[17]} with varying success. However, plasma membrane is essential for cell functions and plasticity, and nanoparticle loading may adversely affect cell signal transduction, adhesion, and migration. According to Irvine et. al.,^{[17,18]}nanoparticles can occupy up to 5% of plasma membrane without significantly affecting cell functions. This translates to a drug loading rate of less than 1.0 μg per million cells. Considering that 1-10 million cells are injected in a normal cell transfer procedure, the amount of drugs that can be delivered using this approach is very limited; not to mention that macrophages are phagocytes, and membrane-bound nanoparticles are often quickly engulfed by cells rather than residing on the surface.

An alternative strategy is to load drugs into the cell cytosol. This approach is considered challenging or not feasible because most chemotherapeutics are highly toxic to macrophages. Incubating macrophages with high concentrations of drugs induces immediate cell death, whilst sub-lethal dose incubation causes insufficient drug loading. What are thus needed are new compositions and methods for increasing drug loading into cells.

The compositions and methods disclosed herein address these and other needs by providing silica-based nanoparticles useful for drug delivery. The nanoparticles can be loaded into cells such as macrophages rather than on the cell surface. This approach facilitates high drug loading, and can target drug delivery to desirable locations in vivo. The nanoparticles can degrade slowly in short-term within macrophages for cells to migrate to desirable locations in vivo, then release the therapeutic or diagnostic agents once reaching the desirable delivery location. The minimal drug release phase can be tuned by adjusting silica nanoparticle compositions.

### SUMMARY

In one aspect, disclosed herein is a nanoparticle comprising: a therapeutic or diagnostic agent positioned within an orthosilicate inner matrix, and a silica outer shell encapsulating the inner orthosilicate-therapeutic/diagnostic matrix.

In another aspect, provided herein are methods of preparing a nanoparticle comprising: combining a therapeutic or diagnostic agent and an orthosilicate to form an orthosilicate inner matrix, and forming a silica outer shell over the inner orthosilicate matrix.

In another aspect, provided herein are methods of treating a disease in a subject comprising administering to the subject a biological carrier cell comprising the disclosed nanoparticles.

In another aspect, provided herein are methods of delivering a therapeutic or diagnostic agent to a target cell comprising contacting the target cell with a biological carrier cell comprising the disclosed nanoparticles. The target cell can be in diseased tissue. In other examples, the target cell can be in healthy tissue.

In another aspect, provided herein are methods of loading a therapeutic or diagnostic agent into a biological carrier cell comprising contacting the biological carrier cell with the disclosed nanoparticles.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying figures, which are incorporated in and constitute a part of this specification, illustrate certain examples of the present disclosure and together with the description, serve to explain, without limitation, the principles of the disclosure. Like numbers represent the same element(s) throughout the figures.

FIGS. 1a through 1d are images and graphs showing the physical characterizations of drug-silica nanocomplex (DSN) nanoparticles. FIG. 1a shows TEM images and FIG. 1b shows zeta potential of DSN-0, DSN-12, DSN-22, and DSN-52 nanoparticles. FIG. 1c shows drug release profiles of DSN-0, DSN-12, DSN-22, and DSN-52 nanoparticles, measured at pH 5.0. FIG. 1d shows TEM images of DSN-52 nanoparticles after incubating in a pH 5.0 solution for different times. Scale bars, 50 nm.

FIGS. 2a through 2i show DSN-52 nanoparticles uptake by macrophages (RAW264.7 cells). FIG. 2a shows intracellular Dox contents, measured at 1, 2, and 4 hours' incubation with DSN-52 nanoparticles. ***, p<0.001. NS, not significant. FIG. 2b shows intracellular Dox contents, measured when the initial DSN-52 Dox concentration was 0, 10, 20, and 40 μg mL⁻¹. The incubation time was fixed at 2 h. FIG. 2c shows cell viability at 12 h via MTT assay. The cells were first incubated with DSN-52 at 0, 10, 20, and 40 μg mL⁻¹ (Dox concentration) for 2 h. After PBS washing, fresh growth medium was added, and cell viability was measured at 12 h by MTT assay. FIG. 2d shows cell viability at 24 h via MTT assay. Dox (black curve), RAW264.7 cells were incubated with free Dox for 24 h. DSN-52 (blue curve), RAW264.7 cells were laden with DSN-52 and then incubated in normal growth medium for 24 h. FIG. 2e shows live and dead cell assay results of DSN-MF and MF cells at 2 h. Green, living cells; red, dead cells. Scale bars, 100 μm. FIG. 2f shows transmigration assay. DSN-MF or MF cells were loaded onto the top of a transwell chamber, whilst U87MG cells were seeded at the bottom. Macrophages were stained into blue color via Giemsa staining Scale bars, 100 μm. FIG. 2g shows fluorescence microscopic images of invaded/migrated DSN-MF cells, the experimental conditions were the same as those in f. Scale bars, 100 μm. Percentages of DSN-MF and MF cells that had (FIG. 2h) migrated and (FIG. 2i) invaded. NS, not significant.

FIGS. 3a through 3k show impact of DSN loading on macrophage phenotypes. Secretion of (FIG. 3a) IL-1β, (FIG. 3b) IL-6, (FIG. 3c) IL-10, (FIG. 3d) IL-12, and (FIG. 3e) TNF-α from DSN-MF at 2 and 24 h. MF (untreated RAW264.7 cells) served as controls (FIG. 3k). FIG. 3f shows IL-12/IL-10 ratio at 2 and 24 h. FIG. 3g shows percentage of Dox released from DSN-MF at different times (Dox retained in cell debris is excluded by centrifugation). FIG. 3h shows cell viability assay results with U87MG cells. Supernatants taken from DSN-MF culture dishes at different time points were added to a separate plate grown with U87MG cells. Cell viability was measured after 48 h incubation. *, P<0.05; **, P<0.01; ***, P<0.001; NS, not significant. FIG. 3i shows hydrodynamic size of exosomes via DLS analysis (z-average size=97.35 nm, PDI=0.127). Exosomes were collected from DSN-MF supernatant at 48 h via centrifugations. An inset photograph of the resulting exosomes and a negative-stained TEM image were also shown. Scale bar, 50 nm. FIG. 3j shows Western blot analysis of exosome lysates. Flotilin-1, TSG101, and CD81, three markers of exosomes, were detected.

FIGS. 4a through 4h show in vivo tumor targeting of DSN-MF, evaluated in nude mice bearing subcutaneously inoculated U87MG tumors (FIG. 4h). FIG. 4a shows axial T2 MR images, acquired at 0, 1, 4, and 24 h post i.v. injection of DSN-MF cells. The cells were pre-loaded with iron oxide nanoparticles. FIG. 4b shows confocal microscopic images of tumor cryo-sections using the z-stack scan mode (step=2 μm). DSN-MF cells were pre-labeled with DiD. Red, DiD; green, Dox; blue, cell nuclei. Scale bars, 50 μm. FIG. 4c shows decay-corrected whole-body coronal PET images, acquired at 1, 8, and 23 h post injection. DSN-MF or MF cells were labeled with 64Cu-PTSM. Tumor area was highlighted with yellow cycles; lung area was highlighted using cyan cycle. Distribution of (FIG. 4d) MF cells and (FIG. 4e) DSN-MF cells in the lung, liver, kidney, and muscle are shown at different time points. FIG. 4f shows tumor uptake of MF and DSN-MF cells at different times. FIG. 4g shows tumor-to-liver ratios of MF and DSN-MF cells, based on images results in FIG. 4c.

FIGS. 5a through 5d show therapy studies with U87MG tumor bearing mice. Animals were randomized to receive one dose i.v. injection of either PBS, free Dox (3 mg Dox kg⁻¹), DSN-52 (3 mg Dox kg⁻¹), RAW264.7 cells (MF, ˜4×10⁶ cells per mouse), or DSN-MF (3 mg Dox kg⁻¹, ˜4×10⁶ cells per mouse). FIG. 5a shows Tumor growth curves. FIG. 5b shows body weight changes. FIG. 5c shows Kaplan-Meier plot of animal survival. FIG. 5d shows in situ apoptosis staining (Abcam) analysis of cryo-sectioned tumor tissues at 24 h post treatments. Cytoplasm region was counterstained into green color by methyl green; nuclei of apoptotic cells were counterstained into dark brown dots by diaminobenzidine. Scale bar, 50 μm.

FIGS. 6a through 6h show results of toxicity studies. FIG. 6a shows animal body weight changes. FIG. 6b shows animal rectal temperature changes. There was a small degree of weight loss in Dox, DSN-52, and DSN-MF group, which was recovered within 5 days. Mice were euthanized on Day 7 for H&E and plasma protein marker analysis: FIG. 6c shows plasma CRP, FIG. 6d shows TNF-α, FIGS. 6e and 6f show AST and ALT levels, respectively, and FIG. 6g shows BUN levels. For DSN-MF, all the indices were in the normal range. FIG. 6h shows H&E staining of major organs, which were collected on Day 7 post treatments. Except for a small degree of elevated leukocyte infiltration, no pathological changes were observed for the DSN-MF group. Scale bar, 100 μm.

FIG. 7 is a schematic showing nanocapsule-laden macrophages for drug delivery to tumors. (1) Antineoplastic drug, in this particular case Dox, was first loaded into a carefully tailored nanocapsule called drug-silica nanocomplex (DSN); (2) DSN nanoparticles were engulfed by macrophages ex vivo; (3) DSN-laden macrophages (DSN-MF) were i.v. injected to a tumor bearing mouse; (4) chemotactic migration of DSN-MF to tumors; (5) DSN-MF releases Dox inside tumor to selectively kill cancer cells.

FIGS. 8a through 8e are graphs and images showing physical and experimental characteristics of the nanoparticles. FIG. 8a shows drug release profiles at pH 7.4 and FIG. 8b shows hydrodynamic sizes of DSN-0, DSN-12, DSN-22, and DSN-52 nanoparticles. FIG. 8c shows digital photograph of DSN-52 nanoparticle dispersed in PBS. FIG. 8d shows TEM images showing DSN-52 nanoparticles' morphology changes over time in a pH 7.4 PBS solution. Scale bar, 50 nm. FIG. 8e shows SEM and elemental mapping by EDS with DSN-52 nanoparticles.

FIGS. 9a and 9b are graphs showing drug release profiles measured at pH 5.0 and 7.4 with (FIG. 9a) Doxove and (FIG. 9b) Dox-encapsulated mesoporous silica nanoparticles.

FIG. 10 is a graph depicting a drug loading assay showing the internalization of DSN-52 nanoparticles into macrophages. Control, DSN-52 nanoparticles were laden into RAW264.7 cells via incubation under normal condition; 4° C., loading was conducted at 4° C.; 4° C.+NaN₃, loading was conducted at 4° C. with the presence of 0.1 wt. % NaN₃. ***, p<0.001.

FIGS. 11a and 11b are graphs depicting results of drug-treated macrophages. FIG. 11a shows intracellular Dox contents. RAW264.7 cells were incubated with Doxove at 20 and 40 μg Dox/mL for 2 or 12 h. FIG. 11b shows cell viability. RAW264.7 cells were incubated with Doxove at different concentrations for 2 h. After replenished with fresh media, the cells were cultured for 24 h, and the viability measured by MTT.

FIGS. 12a through 12c show migration, drug uptake, and drug release in DSN-treated cells. FIG. 12a shows transwell invasion/migration assay results. FIG. 12b shows FL images of U87MG cells after incubating with different supernatants from DSN-MF cell cultures for 6 h. FIG. 12c shows FL spectroscopy analysis of exosome lysates, which confirms the presence of Dox in the exosomes. Excitation was set at 470 nm.

FIG. 13 is an image showing Prussian blue staining on tumor cryo-section. Tumors were collected at 24 h after i.v. injection with IONP-labeled DSN-MF cells.

FIGS. 14a through 14c show results of DSN-treated tumorous mice. FIG. 14a shows Tumor growth curves for individual animals. FIG. 14b shows in situ apoptosis staining analysis of cryo-sectioned tumor tissues. The tumors were dissected 24 h post treatments. The whole tumors were subjected to staining and the positively stained areas quantified by Photoshop. FIG. 14c shows quantitative analysis based on the staining results of FIG. 14b.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nanoparticle is disclosed and discussed and a number of modifications that can be made to the nanoparticle are discussed, specifically contemplated is each and every combination and permutation of the nanoparticle and the modifications that are possible unless specifically indicated to the contrary. Thus, if a class of nanoparticles A, B, and C are disclosed as well as a class of nanoparticles D, E, and F and an example of a combination nanoparticle, or, for example, a combination nanoparticle comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “can,” “may,” “optionally,” “can optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

Use of the phrase “and/or” indicates that any one or any combination of a list of options can be used. For example, “A, B, and/or C” means “A”, or “B”, or “C”, or “A and B”, or “A and C”, or “B and C”, or “A and B and C”.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., rheumatoid arthritis). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of chronic inflammation. Therapeutically effective amounts of a given therapeutic agent will typically vary with respect to factors such as the type and severity of the disorder or disease being treated and the age, gender, weight, and general condition of the subject. Thus, it is not always possible to specify a quantified “therapeutically effective amount.” However, an appropriate “therapeutically effective amount” in any subject case may be determined by one of ordinary skill in the art using routine experimentation. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. It is understood that, unless specifically stated otherwise, a “therapeutically effective amount” of a therapeutic agent can also refer to an amount that is a prophylactically effective amount. In some instances, a desired biological or medical response is achieved following administration of multiple dosages of the composition to the subject over a period of days, weeks, or years.

“Treat,” “treating,” “treatment,” and grammatical variations thereof as used herein, include the administration of a composition with the intent or purpose of partially or completely, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

### Compositions

It is understood that the nanoparticles of the present disclosure can be used in combination with the various compositions, methods, products, and applications disclosed herein.

Disclosed herein is a nanoparticle technology to solve drug and delivery issues such as macrophage drug loading complications. It takes 6-12 h for intravenously (i.v.) injected macrophages to migrate to inflamed tissues.^{[6,24]} If drug-loaded nanoparticles do not release the payloads in the early hours of cell entry, the adverse impacts can be held in check in spite of a high apparent drug content. This would buy time for carrier cells such as macrophages to traffic to tumors, and release therapeutics to induce efficient and selective cancer cell killing (FIG. 7). For this purpose, it is desired that nanoparticles have a two-phase drug release profile, with minimal drug liberation in the first 6-12 h and controlled release afterwards. This is challenging because nanoparticles after internalization are trapped in the phagolysosomes of macrophages, which are rich in hydrolytic enzymes and reactive oxygen species (ROS), and can quickly digest conventional drug carriers.^{[25,26]} To solve the issue, a drug-silica nanocapsule platform is disclosed herein, which contains a drug-silica nanocomplex core, and a solid silica sheath. The silica coating is more resistant to degradation and oxidation than alternative materials such as polymers or liposomes; by fine-tuning the coating thickness, stalled drug released would be achieved and the degree of extended release adjusted. The drug-silica nanocomplex is more susceptible to degradation than the shell as drug molecules create de facto defects in the silica matrix,^{[27]} leading to two-phased drug release. Meanwhile, because drug molecules are electrostatically bound with silica, burst drug release, which is commonly seen with conventional drug carriers, can be avoided. All these properties allow for high drug loading into macrophages while minimally affecting cell migration. Doxorubicin (Dox) was employed as a representative chemotherapeutic drug to demonstrate the efficiency of drug delivery in vitro and in vivo in tumor bearing mice.

Disclosed herein are nanoparticles and nanoparticle compositions comprising one or more diagnostic and/or therapeutic agents. Disclosed is a nanoparticle comprising: a therapeutic or diagnostic agent positioned within an orthosilicate inner matrix, and silica outer shell encapsulating the orthosilicate inner matrix.

The orthosilicate inner matrix comprises a therapeutic or diagnostic agent. The agent can be positioned within the orthosilicate inner matrix such that the orthosilicate inner matrix is more susceptible to degradation in acidic pH or upon administration to a subject than the matrix would be in the absence of the agent. For instance, the agent can alter, disrupt, truncate, make discontinuous, or make irregular the orthosilicate inner matrix. Without wishing to be limited to any particular theory, it is believed that the agent can interfere with the covalent and/or noncovalent chemical bonds within the matrix, thereby weakening the overall bonding strength between components of the matrix and rendering the matrix more susceptible to degradation (e.g., by hydrolysis). The agent can be positioned within the orthosilicate inner matrix by, for instance, being dispersed, embedded, or intercalated within the matrix.

The therapeutic or diagnostic agent generally has a size (e.g., molecular weight) sufficient for positioning within an orthosilicate matrix such that the matrix can still form but has reduced integrity such that the matrix is more susceptible to degradation than in the absence of the agent. In some embodiments, the agent has a molecular weight of 10,000 g/mol or less, 10,000 g/mol or less, 5,000 g/mol or less, 2,500 g/mol or less, 1,000 g/mol or less, 500 g/mol or less, or 100 g/mol or less.

At least one therapeutic or diagnostic agent is positioned within the orthosilicate inner matrix. However, the nanoparticle can comprise more than one agent. In some embodiments, two or more therapeutic or diagnostic agents are positioned within the orthosilicate inner matrix. In some embodiments, three or more, four or more, or five or more therapeutic or diagnostic agents are positioned within the orthosilicate inner matrix. The orthosilicate inner matrix can further comprise additional components which are not diagnostic or therapeutic agents, for instance additional components which modulate the integrity or continuity of the orthosilicate inner matrix.

The therapeutic or diagnostic agent can be any agent capable of being combined within the orthosilicate inner matrix, but is generally an inorganic or biochemical compound. In some embodiments, the therapeutic or diagnostic agent is toxic to a biological carrier cell. In some embodiments comprising a therapeutic agent, a therapeutically effective dose of the therapeutic agent cannot be loaded into or attached to a biological carrier cell for subsequent administration to a subject. In some embodiments, the agent is a therapeutic agent which is toxic or causes side-effects when administered systemically in a therapeutically effective dose to a subject. In some embodiments, the agent can be an anti-cancer chemotherapeutic, an antimicrobial or antibiotic agent, or an anti-inflammatory agent.

Increasing the amount of the agent within the orthosilicate inner matrix can increase the susceptibility of the matrix to hydrolysis. Thus, the ratio of agent to the total amount of SiO₂+agent within the matrix can be an important parameter to adjust the degradation rate of the nanoparticle. In some embodiments, the weight ratio of the agent to the total SiO₂+agent is about 30% or less, about 25% or less, about 20% or less, about 15% or less, about 10% or less, or about 5% or less.

The orthosilicate inner matrix comprises a silica-containing matrix which can degrade at acidic pH (e.g., pH 6.5 or less, pH 6.0 or less, pH 5.5 or less, pH 5.0 or less) or when administered in vivo to a subject. In some embodiments, the orthosilicate inner matrix comprises an orthosilicate attached to one or more organic groups, for example an alkyl group. In some embodiments, the organic group attached to the orthosilicate comprises a C1-C12 group, a C1-C8 group, a C1-C6 group, a C1-C4 group, or a C1-C2 group. In some embodiments, the orthosilicate inner matrix comprises tetramethyl orthosilicate (TMOS), tetraethyl orthosilicate (TEOS), or tetrabutyl orthosilicate (TBOS). In some embodiments, the orthosilicate inner matrix comprises silane derivatives containing functional groups such as amino, thiol, carboxyl, aldehyde, isocyanate, cyano, polyethylene glycol, acetyl, alkyne, alkene, halides, phenol, benzyl, azide, and epoxy, etc. Examples include (3-aminopropyl)-triethoxysilane (APTES), (3-aminopropyl)-diethoxy-methylsilane (APDEMS), (3-mercaptopropyl)-trimethoxysilane (MPTMS), (3-mercaptopropyl)-methyl-dimethoxysilane (MPDMS), and glycidoxy propyl silane, etc.

The orthosilicate inner matrix can have a diameter ranging from 1 nm to 500 nm, from 10 to 250 nm, from 10 nm to 100 nm, or from 20 nm to 50 nm.

The silica outer shell encapsulates the orthosilicate inner matrix. By “encapsulates,” it is meant that the outer shell surrounds, completely or incompletely, the orthosilicate inner matrix. The outer shell can generally protect the inner matrix from exposure to hydrolytic compounds. The outer shell can comprise the same or different orthosilicate material as the orthosilicate inner matrix. In some embodiments, the orthosilicate inner matrix can comprise additional components, or can be substantially free of impurities or embedded/intercalated components. In some embodiments, most or substantially all silicon atoms are covalently linked to adjacent silicon atoms by an oxygen atom within the silica outer shell. A regular, uninterrupted silica outer shell (i.e. solid silica) can contain stronger intermolecular forces than the orthosilicate inner matrix comprising a therapeutic or diagnostic agent. Generally, the silica outer shell is more resistant to acid-mediated degradation than the orthosilicate inner matrix. The thickness of the outer shell can be adjusted to alter the drug release profile of the nanoparticle. For instance, a thicker (i.e. larger diameter) outer shell can require a longer duration of exposure to acid or hydrolytic compounds to degrade under the same conditions (e.g., pH) than a thinner (i.e. smaller diameter) outer shell.

The nanoparticle comprising an inner orthosilicate matrix and an outer silica layer can have a diameter ranging from 10 nm to 1,000 nm, 10 nm to 500 nm, from 10 to 250 nm, from 10 nm to 100 nm, or from 20 nm to 50 nm. In some embodiments, the nanoparticle is negatively charged. The charge of the silica layer can be adjusted by changing the type of types of silane precursors used for inner or outer silica layer formation. For the inner silicate matrix, the type or types of silane precursors will affect the type, amount, and release profiles of therapeutic/diagnostic agents encapsulated into the particles.

The nanoparticle can have an advantageous burst release profile, particularly as compared to drug-loaded liposomes and mesoporous silica nanoparticles. In some embodiments, the silica-based nanoparticle can have a drug release profile of about 50% or less, about 25% or less, about 15% or less, about 10% or less, or about 5% or less drug release in 12 hours in an aqueous solution having a pH of about 5.0.

The nanoparticle can resist acid-mediated degradation, for instance in an aqueous solution at pH 5.0, for at least one hour, at least two hours, at least three hours, at least five hours, at least six hours, at least eight hours, at least ten hours, at least 12 hours, at least 18 hours, or at least 24 hours. However, the nanoparticle is capable of degrading in the presence of low pH (e.g., 5.0) or when administered to a subject inside cells within a time sufficient to deliver the therapeutic or diagnostic agent to a desirable location. Thus, the nanoparticle is capable of controlled release of the therapeutic or diagnostic agent. In some embodiments, the nanoparticle releases the therapeutic or diagnostic agent after eight hours, after ten hours, after twelve hours, after 18 hours, or after 24 hours of exposure to low pH (e.g., 5.0) or after administration to a subject.

Also disclosed are compositions comprising a biological carrier cell comprising the disclosed nanoparticle. The biological carrier cell can be any biological cell capable of internalizing (e.g., by phagocytosis) the nanoparticle, referred to herein as “loading” the cell. The biological carrier cell can, in some embodiments, be a professional phagocyte, for instance a macrophage, neutrophil, monocyte, mast cell, or dendritic cell. In some embodiments the biological carrier cell can be a stem cell, T-cell, epithelial cell, endothelial cell, fibroblast, mesenchymal cell, lymphocyte such as a T-cells or B-cell, erythrocyte, or natural killer cell.

Also disclosed are compositions comprising the nanoparticle, or compositions comprising the nanoparticle within a biological carrier cell, and a pharmaceutically acceptable excipient.

### Methods

Also disclosed herein are methods to produce nanoparticles. Thus, disclosed herein are methods of preparing a nanoparticle comprising combining a therapeutic or diagnostic agent and an orthosilicate to form an orthosilicate inner matrix, and forming a silica outer shell over the orthosilicate inner matrix.

In some embodiments, the orthosilicate inner matrix is formed by mixing the orthosilicate (e.g., TEOS) and the therapeutic or diagnostic agent in a solution. The inner matrix formation can be facilitated by adjusting the pH (e.g. by adding ammonium hydroxide) or the polarity of the solution (e.g. using ethanol rather than aqueous solutions). In some embodiments, more than one therapeutic or diagnostic agent can be combined with the orthosilicate. In some embodiments, the method further comprises adding an alcohol (e.g., methanol, ethanol, propanol, butanol, etc.) to a mixture comprising the therapeutic or diagnostic agent and orthosilicate. In some embodiments, the methods further comprise collecting the formed orthosilicate inner matrix, for instance by centrifugation.

The nanoparticles comprise an silica outer shell which encapsulates the orthosilicate inner matrix. In some embodiments, the outer shell is formed over the orthosilicate inner matrix by combining the orthosilicate inner matrix with the same or a different orthosilicate in a basic solution, which can be an aqueous solution containing ammonium hydroxide. In some embodiments, the method further comprises adding an alcohol (e.g., methanol, ethanol, propanol, butanol, etc.) to the mixture comprising the orthosilicate inner matrix and the added orthosilicate. In some embodiments, the methods further comprise collecting the formed nanoparticle, for instance by centrifugation.

The size (e.g., diameter, molecular weight) of the nanoparticle can be adjusted by adjusting the amount of orthosilicate in the step of forming the inner matrix, the step of forming the outer shell, or both. The size control can also be achieved by adjusting the pH of the solution or the reaction temperature.

In some embodiments, the nanoparticles may be further combined with a biological carrier cell. For example, the nanoparticles can be incubated with a biological carrier cell to facilitate uptake (e.g., by phagocytosis) of the nanoparticle by the carrier cell.

Also disclosed are methods of treating a disease in a subject comprising administering to the subject a biological carrier cell comprising the nanoparticle of claim 1. Generally, the administered biological carrier cell comprising the nanoparticle can deliver the nanoparticle to a desirable location within the subject, for instance a cite of inflammation or tumor.

The administering step can include any method of introducing the nanoparticle into the subject appropriate for the nanoparticle formulation. The administering step can include at least one, two, three, four, five, six, seven, eight, nine, or at least ten dosages. The administering step can be performed before the subject exhibits disease symptoms (e.g., prophylactically), or during or after disease symptoms occur. The administering step can be performed prior to, concurrent with, or subsequent to administration of other agents to the subject. The administering step can be performed with or without co-administration of additional agents (e.g., anti-cancer agents). In some embodiments, the amount of nanoparticles administered (and hence, the amount of therapeutic agent administered) is a therapeutically effective amount.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers.

The disease can be any disease in which administration of the disclosed nanoparticle can be used to treat. The selected therapeutic or diagnostic agent within the nanoparticle typically depends on the disease the nanoparticle is intended to treat or diagnose. In some embodiments, the disease comprises cancer or a tumor. In some embodiments, the disease comprises a chronic inflammatory disease including, for example, arthritis (e.g., osteoarthritis, rheumatoid arthritis, collagen antibody-induced arthritis). In some embodiments, the disease comprises a microbial infection such as Tuberculosis. In some embodiments, the disease comprises a cardiovascular disease such as atherosclerosis or stroke.

Also disclosed are methods of delivering a therapeutic or diagnostic agent to a target cell comprising contacting the target cell with a biological carrier cell comprising the nanoparticles disclosed herein. The biological carrier cell can be any herein disclosed biological carrier cell. In some embodiments, the biological carrier cell comprises one or more receptors which bind to the target cell, or to tissue near the target cell. In some embodiments, the biological carrier cell can translocate or migrate to a medium comprising the target cell, for example a liquid medium having acidic pH. In some embodiments, the biological carrier cell is a macrophage.

In some embodiments, the target cell is a tumor or cancer cell. In some embodiments, the target cell is a microbial cell or a subject's cell infected with a microbial agent.

Also disclosed are methods of loading a therapeutic or diagnostic agent into a biological carrier cell comprising contacting the biological carrier cell with the nanoparticle of claim 1. The biological carrier cell can be contacted with the nanoparticle for any time sufficient to load the nanoparticle into the biological carrier cell, for instance at least one hour, at least two hours, at least five hours, at least eight hours, at least ten hours, at least twelve hours, at least 18 hours, at least 24 hours, at least 2 days, at least 3 days, or at least 7 days. One particular method for loading the nanoparticle into the biological carrier cell comprises incubating the nanoparticle with the biological carrier cell in cell culture.

In some embodiments, a therapeutically effective amount of a therapeutic agent is loaded into the biological carrier cell. In some embodiments, a diagnostically effective amount of a diagnostic agent is loaded into the biological carrier cell. The therapeutic or diagnostic agent can be any herein disclosed therapeutic or diagnostic agent.

### Examples

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1

Methods

Chemicals. Tetraethyl orthosilicate (TEOS, ≥99.0%, Sigma-Aldrich), ammonium hydroxide (28.0-30.0%, Sigma-Aldrich), ethanol (200 proof, Decon Labs, Inc.), doxorubicin hydrochloride salt (Dox. HCl, LC Labs), Doxoves™—stealth liposomal Dox.HCl (2.0 mg mL⁻¹, FormuMax), hexadecyltrimethylammonium bromide (CTAB, ≥99%, Sigma-Aldrich), ethyl acetate (Fisher Scientific, HPLC Grade), HCl (J. T. Baker, 36.5-38%).

DSN synthesis. Doxorubicin (Dox)-encapsulated silica nanocomplex (DSN) was synthesized by a modified procedure based on a previous study (S. Zhang, et al., J. Am. Chem. Soc. 2013, 135, 5709). Different volumes of 4.2-4.5% ammonium hydroxide aqueous solutions (e.g., 0.15 mL, 0.3 mL, 0.6 mL) and TEOS (e.g., 2.5, 5, and 10 μL) were added into ethanol to form a 4.0 mL Dox solution of varied concentrations (e.g., 0.10, 0.25, and 0.50 mg mL⁻¹). The mixture was magnetically stirred at room temperature for 24 hours. The drug-loaded NPs were collected by repeated washes with ethanol and centrifugation (12,000 rpm, 5 min). The as-synthesized particles were lyophilized and stored at −80° C. in the dark. Specifically, DSN-0 NP was synthesized using 0.3 mL ammonium hydroxide, 5 μL TEOS, and 0.25 mg Dox mL⁻¹. For silica coating onto DSN-0, the as-synthesized DSN-0 was re-dispersed in 4.0 mL 200 proof ethanol with brief sonication. Then, 0.3 mL ammonium hydroxide solution (4.2-4.5%) and different volumes of TEOS (e.g., 2.5, 5.0, and 10.0 μL) were added dropwise to the colloidal solution. The mixture was magnetically stirred at room temperature for 24 hours. The coated DSN nanoparticles were washed, collected, lyophilized, and stored at −80° C. following the same protocol. According to the coating thicknesses measured by TEM, the coated DSNs were designated as DSN-12, DSN-22, and DSN-52, respectively.

Mesoporous silica nanoparticle synthesis. Mesoporous silica nanoparticles were synthesized following a published protocol (W. X. Mai, et al., Integr. Biol. (United Kingdom) 2013, 5, 19). Briefly, nanoparticles were prepared by mixing 3 mL TEOS with CTAB (5.5 mM) in a 300 mL, 70° C. aqueous solution containing 4.2 mmol NaOH, followed with the addition of 18 mL ethyl acetate. Free CTAB was removed by stirring nanoparticles in 100 mL ethanol containing 1 mL 37% HCl at 60° C. for 3 hours. The as-synthesized nanoparticles were dried at 60° C. overnight. To load Dox, mesoporous silica nanoparticles were stirred in a Dox ethanol solution (2.5 mg mL⁻¹) overnight at room temperature in the dark. The resulting Dox-encapsulated NPs were washed with water twice, lyophilized, and stored at −80° C.

Characterization of nanoparticles. The morphology, size distributions, zeta potential, and EDS of nanoparticles were characterized by transmission electron microscope (TEM, H-9500), scanning electron microscope (SEM, FBI Teneo), and dynamic light scattering (DLS, Malvern Zetasizer Nano S90). The temporal degradation of nanoparticles at pH 5.0 and 7.4 was examined using TEM. Briefly, nanoparticles dispersed in PBS (pH 5.0 and 7.4) were incubated at 37° C. under constant shaking for 2, 6, 24, and 72 h. The remaining nanoparticles were collected and examined under TEM for morphology and size changes. Absorbance at 470, 480, 490 nm was used to quantify Dox content. The loading capacity (% LC) was calculated by the following equation: % LC=(Drug loaded)/(nanoparticle weight)×100%, where the amount of drug loaded was determined by absorbance, and the nanoparticle weight determined either by directly weighing lyophilized nanoparticles or calculating the silica weight based on inductively coupled plasma-optical emission spectrometry (ICP-OES) results. In the latter case, it was assumed that silica dioxide (SiO₂) was the major silica component and that nanoparticle weight=Dox weight+SiO₂ weight. Drug release of different nanoparticle formulations was determined using a Slide-A-Lyzer 10K MWCO mini dialysis device (Thermo Scientific). Briefly, nanoparticles containing the same Dox content were dispersed in 0.5 mL PBS and dialyzed against 14 mL PBS (pH 5.0 and 7.4) at 37° C. under constant shaking. At different time points (i.e., 0, 0.5, 1, 2, 4, 8, 12. 24, 48, 72, 96, 120 hour), a 0.5 mL PBS sample from the bottom chamber was collected, which was supplanted with 0.5 mL fresh PBS. Cumulative Dox release over 5 days was quantified by subtracting the remaining Dox in the cassette from the initial loading amount. Dox concentration in the sample solutions were measured by fluorescence spectroscopy analysis (ex/em: 470/590 nm).

In vitro cellular loading studies. RAW264.7 (murine macrophages) and U87MG (human glioblastoma) were purchased from ATCC. RAW264.7 cells were cultured in RPMI1640 medium (Corning, USA) supplemented with 10% FBS (Corning, USA) and 1% penicillin-streptomycin (MediaTech, USA). During the nanoparticle loading stage, FBS-free RPMI1640 medium was used for culturing. U87MG cells were grown in DMEM medium (Corning, USA) supplemented with 10% FBS, 1% non-essential amino acids, and 1% penicillin-streptomycin. These two cell lines were incubated under 37° C. and 5% CO₂ in a humid chamber. For nanoparticle loading studies, nanoparticles (DSN-22 or DSN-52) of different concentrations (i.e., 0, 10, 20, and 40 μg Dox mL⁻¹) were incubated with RAW264.7 cells for 1, 2, and 4 h, followed with gentle wash with PBS or complete medium. Depending on the purpose of each study, the DSN-laden macrophages were either collected using trypsin treatment or cultured further with complete growth medium. To determine the amount of Dox loaded into cells, DSN-laden cells were counted and then lysed by sonication in PBS (pH 5.0). The amounts of released Dox was measured by spectroscopic analysis. The Dox content on a per cell basis was calculated compared with macrophages without nanoparticle loading. To study whether DSN-52 nanoparticles were laden into cells via internalization, nanoparticles were incubated with RAW264.7 cells under the same condition described above except using 4° C. for incubation and adding 0.1 wt. % NaN₃ (to minimize energy-consuming internalization process). The Dox loading amount on a per cell basis was compared with control. To evaluate the impact of loaded DSNs on macrophages, the viability change was first assessed at different time points post nanoparticle loading by 3-(4,5-dimethythiazon-2-yl)-2,5-diphenyl tetrazolium bromide (MTT) assay or Live/Dead Cytotoxicity assay and the results were compared with normal macrophages. In the end, DSN-52 NPs were selected for cell loading, and the loading process was accomplished by 2-h incubation at a concentration of 20 μg Dox mL⁻¹. All in vitro experiments were repeated at least twice.

Cell invasion/migration assay. Cell invasion/migration assay was used to examine whether DSN-laden RAW264.7 (DSN-MF) cells remained tumor-tropic. Unladen RAW264.7 (MF) cells served as controls. A transwell polycarbonate membrane cell culture insert set (Corning, 8.0 μm pore sized) was fitted into a 6-well cell culture plate for this study. Migration assay involved coating the upper surface of the inserts with a layer of Matrigel beforehand. For experimental groups, U87MG cells (0.2 million cells) as a lure were seeded to the bottom of each well and cultured overnight (Table 1). For the control group, only medium was added into each bottom well. Then, 0.4 million DSN-MF cells or normal RAW264.7 cells (dispersed in 1.0 mL FBS-free RPMI1640 medium) were seeded onto the upper chamber of each insert. The transmigration process took 16 hours to accomplish under normal incubation conditions (37° C., 5% CO₂). Afterwards, the transwell inserts were collected, washed twice with PBS, fixed with formaldehyde (3.7% in PBS) for 2 min, washed twice with PBS again, permeabilized with methanol for 20 min, and subjected to Giemsa staining. Those cells that failed to transmigrate (remained on top of the film) were scraped off with cotton swabs. Optical and fluorescence images of the transmigrated RAW264.7 cells were captured. Due to Giemsa staining, invaded/migrated cells were blue in bright-field images. In fluorescence images, DSN-MF cells were visualized due to the intrinsic fluorescent properties of Dox. For each sample, 25 images of different areas were acquired for cell counting to obtain a statistically significant result. The experiment was repeated twice.

TABLE 1
Cell migration assay conditions.

| # | Upper chamber | Assay Type | Lower chamber |
|---|---|---|---|
| 1 | MF | Invasion | U87MG cells |
| 2 | | | Empty |
| 3 | | Migration | U87MG cells |
| 4 | | (+Matrigel) | Empty |
| 5 | DSN-MF | Invasion | U87MG cells |
| 6 | | | Empty |
| 7 | | Migration | U87MG cells |
| 8 | | (+Matrigel) | Empty |

Cell phenotype change study. Enzyme-linked immunosorbent assay (ELISA) were used to examine the cytokine released by macrophages after they were loaded with DSN-52 nanoparticles. Briefly, the medium supernatants from DSN-MF seeded plates were collected at different time points (e.g., 2 and 24 h post loading) to quantify the concentrations of different cytokines, including interleukin-1beta (IL-1β), IL-6, IL-10, IL-12p70, and tumor necrosis factor-α (TNF-α). The results were compared with normal macrophage control groups with the same cell numbers, the same culture medium volumes, and the same culturing conditions. The experiments were conducted by following vendor-provided protocols (RayBiotech) and the concentrations of each cytokine were calculated by comparing to standard calibration curves. All ELISA tests were repeated at least twice.

In vitro therapy study. DSN-MF as well as MF cells were cultured with complete growth medium for 12, 24, and 48 hours (2 million cells; 5 mL medium for each group). Supernatants from each group were collected. The amounts of Dox in the supernatants were assessed by fluorescence spectroscopy analysis with the help of standard calibration curves. To evaluate the cytotoxicity to cancer cells, U87MG cells that were pre-cultured in a separate 6 well plate overnight (confluence ˜0.4 million cells per well). Supernatants taken DSN-MF cell cultures at 12, 24 h, and 48 h were added into the U87MG cell culture medium. For controls, 1.0 mL complete RPMI1640 medium was added. The viability of U87MG cells at 48 h was examined by MTT assay. For imaging studies to visualize the accumulation of Dox in cancer cells, U87MG cells were co-incubated with different supernatant medium for 6 h, washed with PBS, and then imaged under a fluorescence microscope.

Exosome isolation and analysis. Ten T75 flasks were each seeded with 2 million RAW264.7 cells. After overnight culturing, cells were laden with DSN-52 nanoparticles following above mentioned protocol. The medium supernatant was collected at 45 h post loading. Exosomes in the supernatant were enriched via a series of centrifugation: (1) centrifugation at 300×g for 10 min at 4° C. to remove the living cells, (2) 2000×g at 4° C. for 10 min to remove dead cells, (3) 10,000×g at 4° C. for 30 min to remove the cell debris, (4) a ultracentrifugation step at 100 000×g at 4° C. for 90 min, and (5) 100 000×g at 4° C. for 60 min after PBS wash. Collected exosomes were dispersed in (1) PBS, (2) DI water, and (3) radioimmunoprecipitation assay (RIPA) buffer. For TEM imaging, 5 μL exosome dispersion in DI water was dropped onto a TEM grid and air-dried for 10 min, followed by addition of 5 μL of 1 wt. % uranyl acetate in DI water for negative staining. The hydrodynamic size of exosomes in PBS was analyzed by DLS after filtration twice through a 0.45 μm filter unit. The amount of Dox in exosome was quantified by measuring Abs at 470 nm and comparing to a calibration curve. Alternatively, Dox was quantified by measuring fluorescence with excitation at 470 nm and emission at 590 nm. The protein concentration of exosome lysates was determined by DC protein assay (Bio-Rad Laboratories). Standards and samples (5 μL) were added into a 96-well plate, followed by addition of 25 μL reagent A:S at a ratio of 50:1, and 200 μL of reagent C. The absorbance at 750 nm was measured after 15 min incubation at room temperature. The concentration of exosome lysates was calculated based on a standard calibration curve. For western blotting analysis, the collected exosomes were lysed in RIPA buffer containing protease inhibitor (1×). After denaturing at 95° for 5 min, the lysate was resolved in SDS-PAGE gel and transferred onto nitrocellulose membrane, followed by incubation with primary antibody (1:1000 dilution) at 4° C. overnight and secondary antibody (1:5000 dilution) at room temperature for 1 h. The blot was imaged using enhanced chemiluminescence (ECL).

Small animal models. For imaging and therapy studies, a U87MG subcutaneous mouse model was used. The animal model was established by subcutaneously inoculating 1 million U87MG cells onto the right hind leg or the right flank (for PET imaging only) of a 5-6 week old athymic female nude mice (Harlan). For toxicity studies, normal 5-6 week old balb/c mice (Envigo laboratories). All the animal studied were performed according to a protocol approved by the Institutional Animal Care and Use Committee (IACUC) of University of Georgia.

In vivo MRI and Prussian blue staining. Human serum albumin decorated iron oxide nanoparticles (HSA-IONPs) were prepared according to a previously published protocol (J. Xie, et al., Chem. Commun. (Camb). 2010, 46, 433). HSA-IONPs (20 μg Fe mL⁻¹) and DSN-52 (20 μg Dox mL⁻¹) were co-incubated with RAW264.7 cells for 2 h for cell labeling. The resulting, IONP labeled cells at a dose of around 2 million cells per mouse were i.v. administrated to nude mice bearing U87MG tumors on the right hind leg (tumor size 200 mm³). The mice were scanned on a 7.0 T Varian small animal MRI system before cell injection, as well as 1, 4 and 24 h after the administration. The scan parameters were the following: TR=2500 ms, TE=40 ms, field-of-view (FOV)=40 mm×80 mm, matrix size=2562, thickness=2 mm After the 24-h scan, the mice were euthanized. The liver, spleen, lung, heart, kidney, brain, and tumor tissues were collected and frozen in optical cutting temperature (OCT) compound gel at −80° C. for Prussian Blue staining purpose. The tissue blocks were cryo-sectioned into 8 μm thick slices and fixed in formalin solutions for 10 min The slides were carefully rinsed with PBS twice and then submerged in a solution containing 20% HCl and 10% K₄[Fe(CN)₆].3H₂O for 20 min (Prussian Blue Staining) Afterwards, the slices were washed twice with PBS and counter-stained with Fast Red for 5 min, followed by PBS wash.

Small-animal Positron Emission Tomography. Small-animal PET was performed on a micro-PET R4 scanner. U87MG tumors were inoculated to the right flanks of the nude mice instead of their hind legs to minimize the impact from tracer uptake in the abdomen. Imaging started once the tumor size reached 50-100 mm³. DSN-MF and MF cells were co-incubated with ⁶⁴Cu-pyruvaldehyde-bis(N4-methylthiosemicarbazone) (⁶⁴Cu-PTSM) in 1 mL serum-free medium at 37° C. for 1.5 hours. After washing, 1 million of ⁶⁴Cu-labelded cells in 0.25 mL PBS (pH 7.4) were i.v. injected into each mouse under isofluorane anesthesia. Static scans were performed at various time points (e.g., 1, 8, and 23 h after the injection). The average radioactivities accumulated within the tumor and other major organs were quantified from decay-corrected coronal images and the results were converted to percentage injected dose per gram (% ID g⁻¹).

In vivo therapy study. Treatments started when tumor size reached 100-150 mm³. 25 mice were randomly divided into 5 groups and were i.v. injected with PBS, free Dox, DSN-52 in PBS, MF cells, and DSN-MF cells on Day 0. Dox, DSN-52, and DSN-MF were injected at 3 mg Dox kg⁻¹, and 4 million cells were injected into the mice in MF and DSN-MF groups. The body weight and tumor volume of each mouse were measured every other day for 2 weeks. The tumor volume was calculated by the following equation: tumor volume=0.5×length×(width)², where length≥width. Mice were euthanized once the tumor volume was above 1,700 mm³.

In separate studies, animals were euthanized 24 h after cell/drug injection, and the tumors were collected and frozen in OCT compound gel at −80° C. The tissues were cryo-sectioned into 8 μm thick slices for in situ apoptosis detection staining (ab206386 from abcam) following the vendor's protocol. The apoptotic nuclei were stained as dark brown and the cytoplasm components were green.

Toxicity studies. Fifteen normal balb/c mice were randomly divided into 5 groups and received regimens specified in the in vivo therapy study section. The body weight and anal temperature of each mouse were measured daily at the same time (starting from 6 days before injection through Day 6). On Day 7, all mice were euthanized and the whole blood was collected. Part of the blood samples were used for a complete blood count (CBC) test. The rest were centrifuged at 5000 rcf for 5 min and the resulting serum samples were stored at −80° C. and then subjected to ELISA or colorimetric assays to quantify C-reactive protein (CRP), TNF-α, alanine transaminase (ALT), aspartate aminotransferase (AST), and blood urea nitrogen (BUN) amounts. All tests were conducted by following the vendors' protocols. CRP and TNF-α kits were purchased from RayBiotech, ALT (MAK055) and AST (MAK052) kits from Sigma-Aldrich, and BUN from Arbor Assays. Each assay was repeated at least twice.

Statistical methods. Quantitative data were expressed as mean±SEM. Two-tailed Student's t-test and Chi-squared test were used for statistical comparison between experimental groups and control groups for different studies. P<0.05 was considered statistically significant.

Estimation of drug loading capacity via surface backpack strategy. The number of cells injected during one transfer procedure was calculated to evaluate the drug loading capacity of nanoparticle-laden cell system via surface backpack strategy. Three types of nanoparticles commonly used for drug loading were considered: (1) poly(lactic-co-glycolic acid) (PLGA) nanoparticle; (2) lipid-type nanoparticle, such as a liposome; (3) Stöber silica nanoparticle. For each particle, three parameters were considered for the estimation: (a) the amount drug in each particle, (b) the number of nanoparticles that are tethered on surface of each cell, and (c) the injection dose. The following equation was used to estimate the drug loading amount per nanoparticle: drug loading by weight=% Loading Capacity×nanoparticle weight. Except some rare examples (J. Della Rocca, et al., Angew. Chemie—Int. Ed. 2011, 50, 10330), the % Loading Capacity by weight of PLGA nanoparticle, lipid nanoparticle, and Stöber silica nanoparticle was typically lower than or close to 20% (P. Kan, et al., J. Drug Deliv. 2011, 2011, 1; D. J. A. Crommelin, et al., Int. J. Pharm. 1983, 17, 135; X. Song, et al., Eur. J. Pharm. Biopharm. 2008, 69, 445; M. García-Díaz, et al., Int. J. Pharm. 2015, 482, 84). The nanoparticle weight can be calculated by mass=density×volume. The density of PLGA nanoparticle, lipid nanoparticle, and Stöber silica nanoparticle was around 1.3 g cm⁻³, 1.06 g cm⁻³, and 1.8-2.2 g cm⁻³ respectively. Assuming each nanoparticle was a perfect sphere, the volume of a single nanoparticle equals to (4/3)πr³, where r is the radius of nanoparticles, typically ranging from 100 to 200 nm. Attachment of up to 100-150 nanoparticles with a diameter of ˜200 nm onto the plasma membrane is benign to T cells and hematopoietic stem cells (M. T. Stephan, et al., Nat. Med. 2010, 16, 1035). A clinically relevant treatment dose usually ranges from 1 to 10 mg kg⁻¹.

To simplify the calculation, drug encapsulated nanoparticles were assumed as a homogeneous entity with a fixed density, the % Loading Capacity=20%, and the number of nanoparticles attached onto each single cell=300. At 1 mg kg⁻¹, which is at the low end of typical drug injection dose, and assuming a body weight of 25 g for mouse and 50 kg for human, the amount of drug injected was 25 μg per mouse and 50 mg per person. As shown in Table 2, the drug loading capacity on a per cell basis was estimated to be about 0.03-0.50 pg drug cell⁻¹, which required injection of tens to hundreds of millions of cells per mouse to achieve the 1 mg kg⁻¹ dose. In a recent report about using nanoparticle-carrying T cells for drug delivery (B. Huang, et al., Sci. TransL Med. 2015, 7, 291ra94), the drug loading was 0.1-0.125 pg drug cell⁻¹, which coincided well with estimations calculated here.

TABLE 2
Estimation of drug loading per cell via the surface backpack strategy.

| | | | | | drug per | drug per | #cell per | #cell per |
|---|---|---|---|---|---|---|---|---|
| | density | radius | volume | NP mass | NP | cell | mouse | person |
| NP type | g cm⁻³ | nm | cm³ | pg | pg NP⁻¹ | pg cell⁻¹ | million | billion |
| PLGA | 1.30 | 50 | 0.52 × 10⁻¹⁵ | 0.68 × 10⁻³ | 1.36 × 10⁻⁴ | 0.04 | 612 | 1224 |
| | | 100 | 4.19 × 10⁻¹⁵ | 5.45 × 10⁻³ | 10.89 × 10⁻⁴ | 0.33 | 77 | 153 |
| Lipid | 1.06 | 50 | 0.52 × 10⁻¹⁵ | 0.56 × 10⁻³ | 1.11 × 10⁻⁴ | 0.03 | 751 | 1501 |
| | | 100 | 4.19 × 10⁻¹⁵ | 4.44 × 10⁻³ | 8.88 × 10⁻⁴ | 0.27 | 94 | 188 |
| Stöber Si | 2.00 | 50 | 0.52 × 10⁻¹⁵ | 1.05 × 10⁻³ | 2.09 × 10⁻⁴ | 0.06 | 398 | 796 |
| | | 100 | 4.19 × 10⁻¹⁵ | 8.38 × 10⁻³ | 16.76 × 10⁻⁴ | 0.50 | 50 | 99 |

NP: nanoparticle

Results

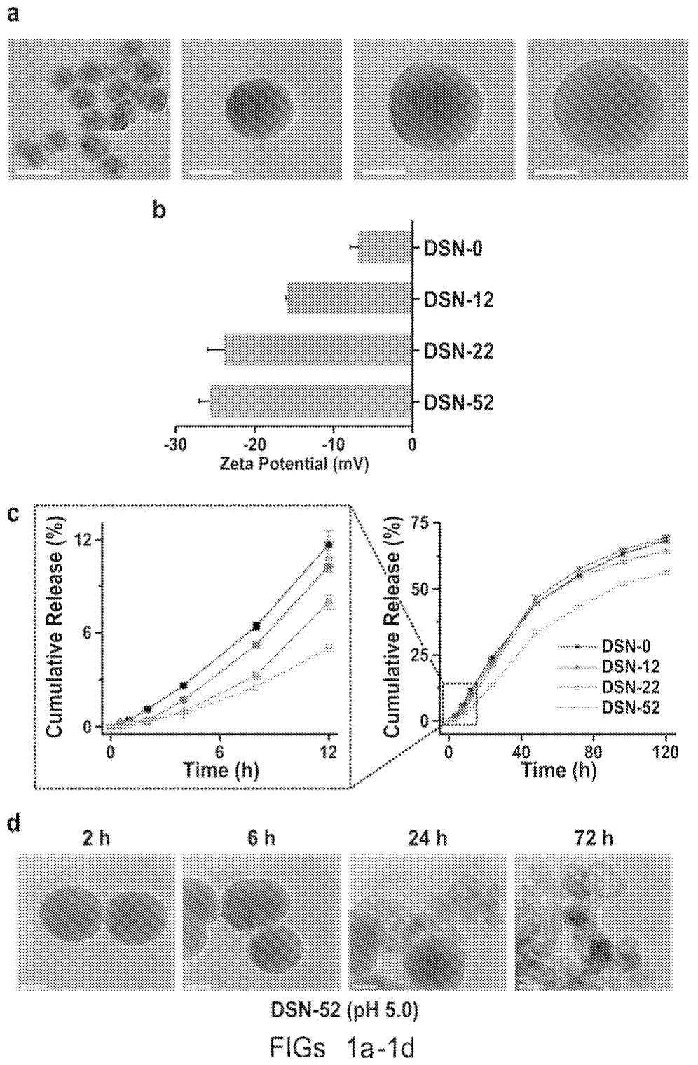

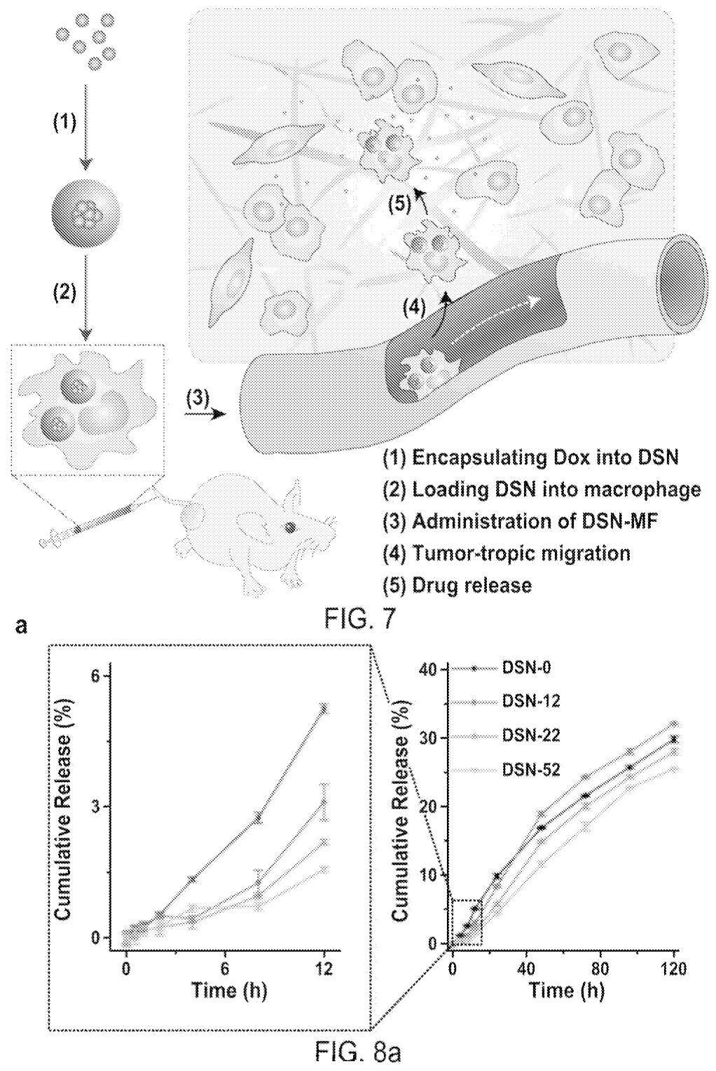

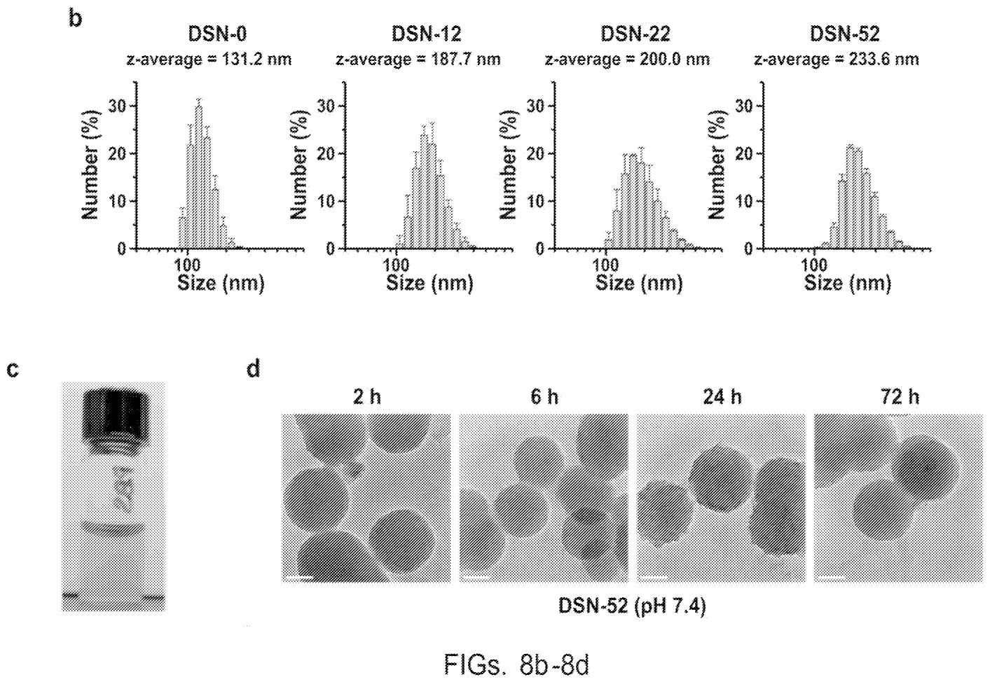

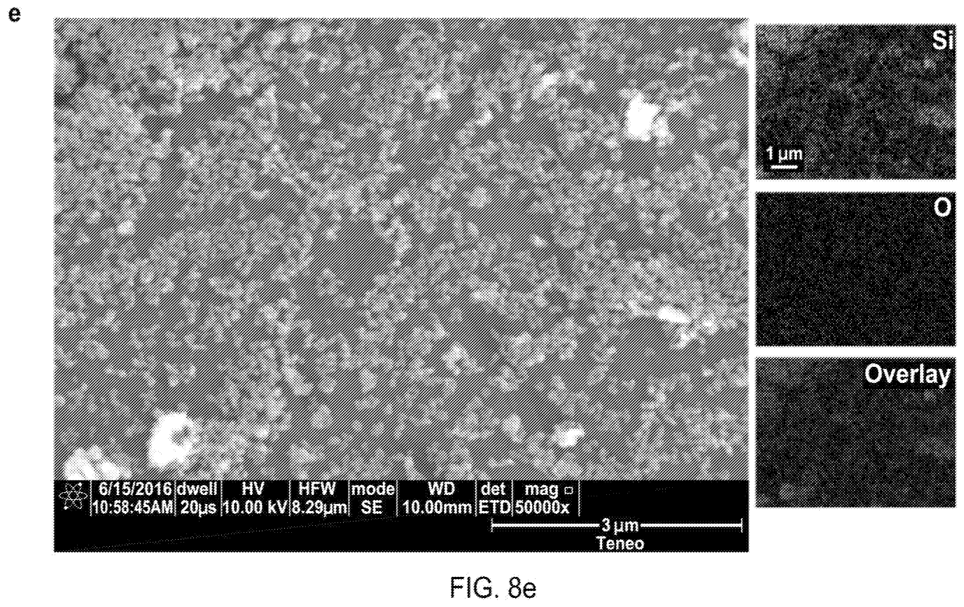

DSN synthesis and physical characterizations. Doxorubicin (Dox)-silica nanocomplexes (referred to as DSN-0) were synthesized by co-condensation of Dox and tetraethyl orthosilicate (TEOS, Dox: TEOS molar ratio of 1:13) in ethanol. Dox is positively charged at this condition and electrostatically bound with TEOS, whilst being intercalated into the growing silica matrix.^{[27]} The resulting DSN-0 nanoparticles were 28.4±3.4 nm in diameter (FIG. 1a). They were stably dispersed in PBS (FIGS. 8b and 8c), with a slightly negatively charged surface (−6.9±1.0 mV; FIG. 1b). Based on UV-Vis spectroscopic analysis, it was estimated that the Dox accounted for 16.7 wt % of the DSN-0 weight (Table 3).

TABLE 3
Loading capacity (% LC) of DSNs

| | Total Wt. | Dox | SiO₂ | Dox Ratio |
|---|---|---|---|---|
| | mg | mg | mg | wt % |
| DSN-0 | 5.0 | 0.83 | 4.17 | 16.70% |
| DSN-12 | 2.6 | 0.29 | 2.39 | 11.15% |
| DSN-22 | 4.7 | 0.42 | 8.34 | 8.94% |
| DSN-52 | 6.7 | 0.34 | 6.36 | 5.13% |

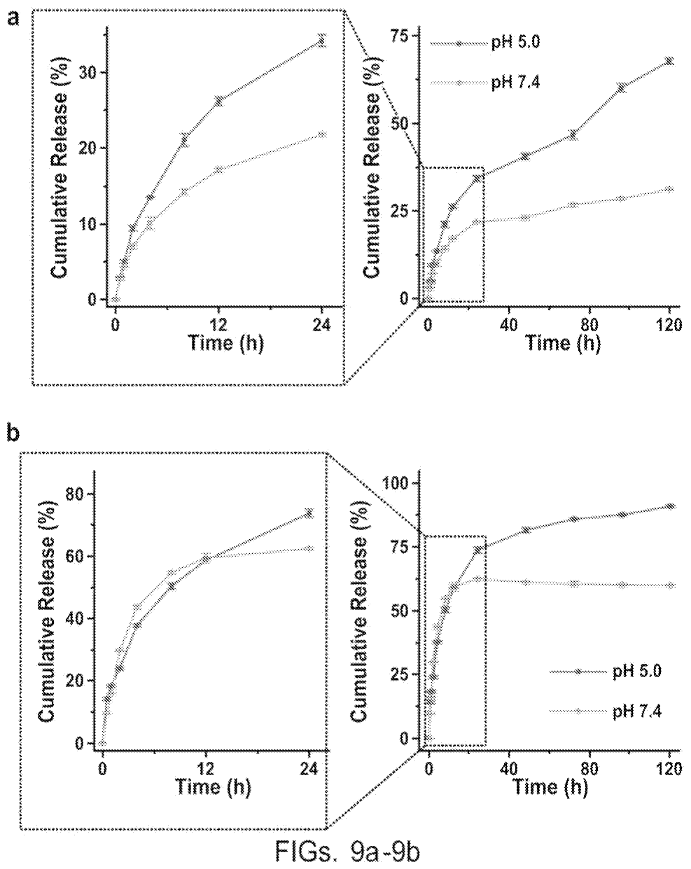

Despite the strong electrostatic interaction, up to 11.7% of Dox was released within 12 h at pH 5.0 (close to lysosome pH^{[28]}; FIG. 1c). To minimize drug release in the early hours, a silica capsule was imparted onto the surface of DSN-0 through the Stöber method. By varying the TEOS precursor amounts, DSN nanocapsules were prepared with silica coating thicknesses of 12, 22, and 52 nm (FIG. 1a), and the resulting nanoparticles were referred to as DSN-12, DSN-22, and DSN-52, respectively. A thicker silica coating was associated with more negative surface charge (FIG. 1b) and more extended drug release (FIG. 1c, FIG. 8a). Specifically, DSN-12 and DSN-22 released 10.3% and 8.0% of their Dox contents at 12 h (pH 5.0), and for DSN-52, this number was reduced to 5.1%. DSN-52's morphology changes over time was investigated in both neutral and acidic solutions. At pH 7.4, DSN-52 remained intact for over 72 h (FIG. 8d). At pH 5.0, on the other hand, no obvious morphology changes in the first 6 h were observed, but signs of nanoparticle degradation occurred at 24 h (FIG. 1d). The gradual erosion of the silica coating exposed the DSN core. Unlike solid silica, where adjacent silicon atom is covalently linked by an oxygen bridge, the silica matrix of DSN is more susceptible to hydrolysis due to the Dox dopants. In samples taken at later time points of incubation, many hollow capsules were observed, suggesting the degradation of the DSN cores (FIG. 1d). This correlated with faster drug release recorded after 12 h. For comparison, drug release was also assessed with Dox-loaded liposomes (Doxove) and mesoporous silica nanoparticles at the same condition. In both cases, significant early-hour burst release was observed, with 26.2% (Doxove, FIGS. 9a) and 58.6% (mesoporous silica nanoparticle, FIG. 9b) of their Dox contents liberated at 12 h, respectively.

Imparting the silica coating diluted the drug content in the nanoparticles. Specifically, the Dox loading was 11.2, 8.9, and 5.1 wt %, respectively, for DSN-12, DSN-22, and DSN-52, compared to 16.7 wt % for DSN-0 (Table 3, Table 4). While it is possible to further increase the silica coating thickness and stall the drug release process, it is speculated that a too diluted drug content in the particles (e.g., less than 5%) may adversely affect Dox loading into macrophages. Due to this consideration, the DSN-52 formulation was selected for subsequent cell and animal studies.

TABLE 4
Loading capacity (% LC) of DSN-52 by ICP-OES.

| | Dox | Si | Soln. | Si | Si | SiO2 | Total | Dox Ratio |
|---|---|---|---|---|---|---|---|---|
| | μg | ppm | g | ppm | μg | μg | μg | wt % |
| BKG | 0 | 0.97 | 5.03 | 0 | 0 | 0 | 0 | |
| #1 | 6 | 10.51 | (5 mL) | 9.54 | 47.986 | 102.8271 | 108.8271 | 5.51% |
| #2 | 12 | 22.4 | | 21.43 | 107.793 | 230.985 | 242.985 | 4.94% |
| | | | | | | | average | 5.23% |

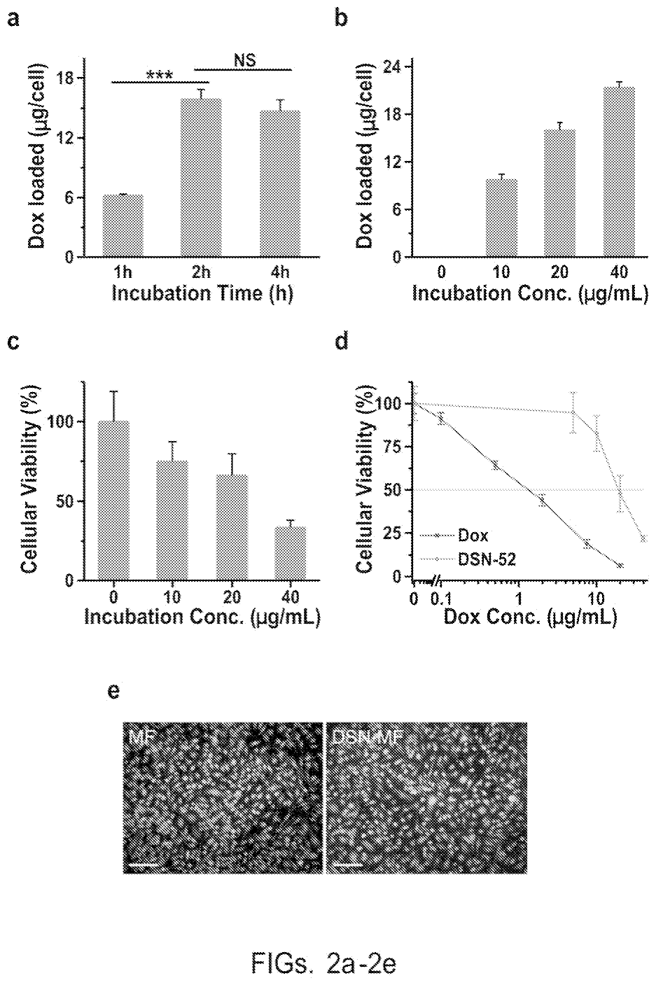

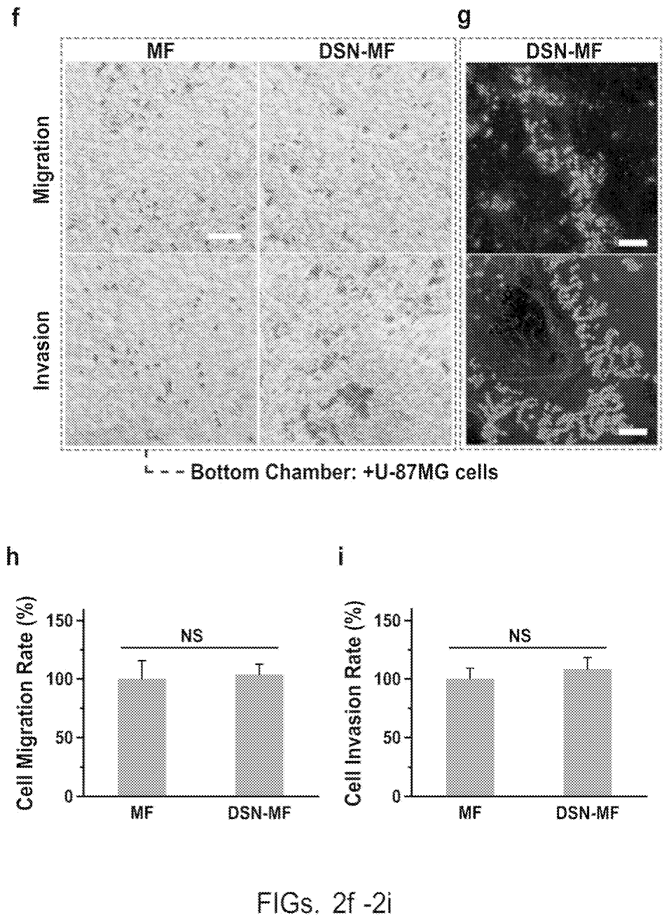

Loading DSN-52 nanocapsules into macrophages. DSN-52 were incubated with RAW264.7 cells, a murine macrophage cell line, and stopped the incubation at different times to analyze nanocapsule uptake by measuring the amount of cellular Dox on a per cell basis. 2 h incubation led to efficient Dox uptake, while extending incubation further minimally increased the cellular Dox contents (FIG. 2a). The uptake was attributed to macrophage phagocytosis of nanoparticles, which was observed by others with nanoparticles of comparable sizes.^{[22]} The uptake was concentration dependent. When incubated with DSN-52 at 10, 20, and 40 μg Dox mL⁻¹, the cell Dox content at 2 h was 9.8, 15.9, and 21.3 pg cell⁻¹, respectively (FIG. 2b). As a comparison, Doxove showed a Dox loading of less than 1 pg cell⁻¹ at the same conditions (FIG. 11a).

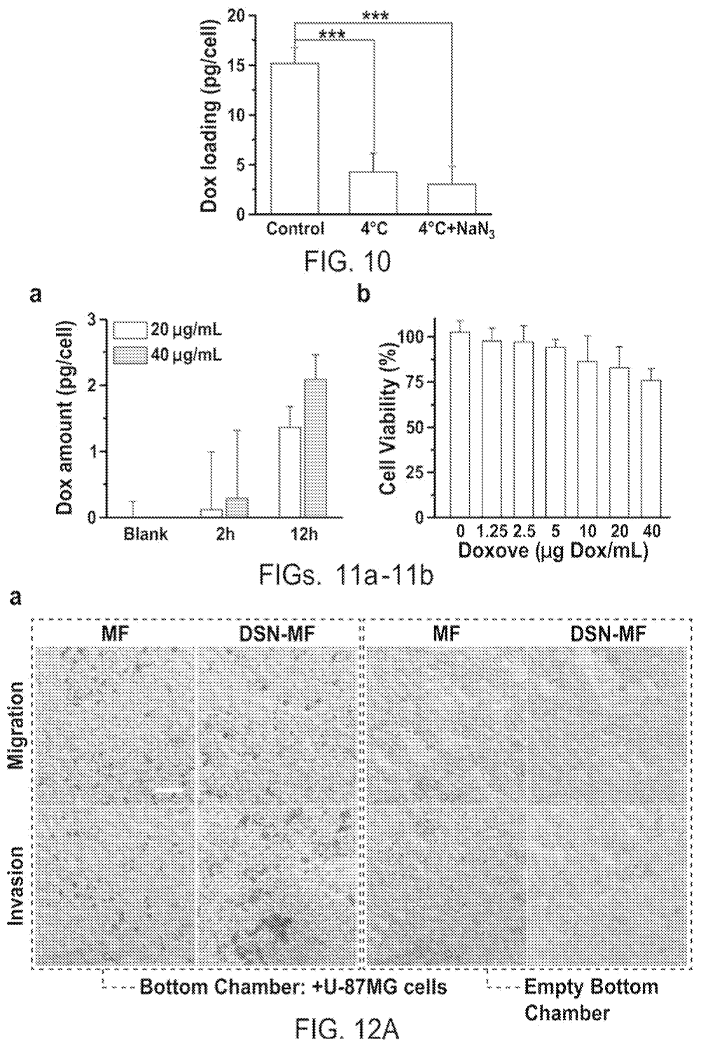

After 2 h incubation, the incubation medium was replenished and the viability of the DSN-laden cells was analyzed. When initial DSN-52 concentration was 20 μg Dox mL⁻¹ or below, cell viability maintained at −70% or above at 12 h (FIG. 2c). This is striking considering that the IC₅₀ of free Dox is 1.5 μg mL⁻¹ (FIG. 2d). Based on these observations, 20 μg Dox mL⁻¹ and 2 h incubation were selected for drug loading. Under these conditions, macrophages bore a stunning Dox content at 16.6±4.8 pg Dox cell⁻¹. Calcein AM/EthD-III assay showed that 99.2% of DSN-52 loaded macrophages (DSN-MF) were heathy at the completion of nanoparticle incubation (FIG. 2e). Incubation at 4° C. in the presence of 0.1 wt. % NaN₃ led to an about 80% decrease of cellular uptake, suggesting that the nanoparticle uptake was mainly mediated by endocytosis (FIG. 10).

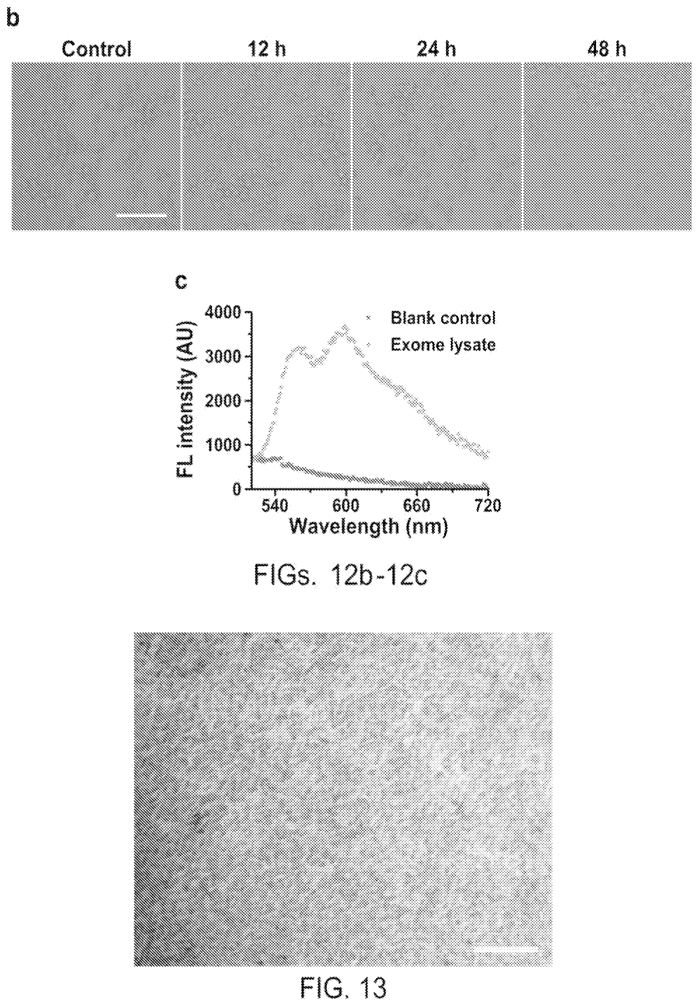

One potential concern is that the intracellular Dox, while not lethal, may affect cell functions. In particular, the drug-nanoparticle loading may potentially compromise cells' chemotactic migration toward cancer cells. This was examined in a transwell experiment, where U87MG glioblastoma cells were seeded onto the bottom chamber of the device, and DSN-MF loaded onto the top. DSN-MF could efficiently transmigrate the well (FIGS. 2f and 2g), with both invasion and migration percentages comparable to untreated RAW264.7 cells (referred to herein as “MF”; FIG. 2h and FIG. 2i). Meanwhile, when U87MG cells were absent, there was no cell transmigration (FIG. 12a).

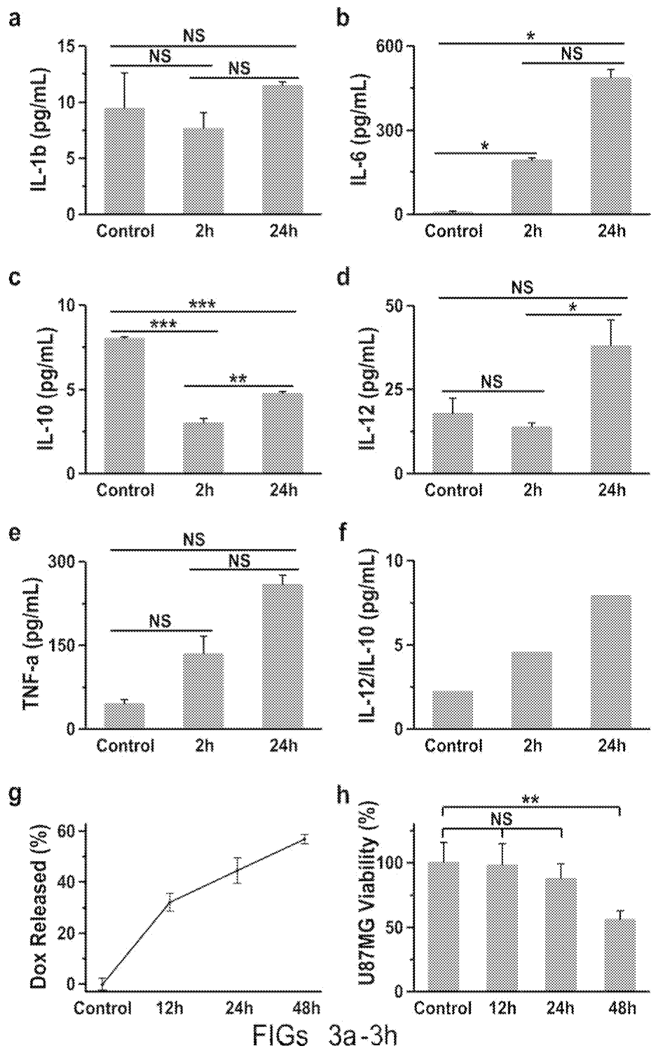

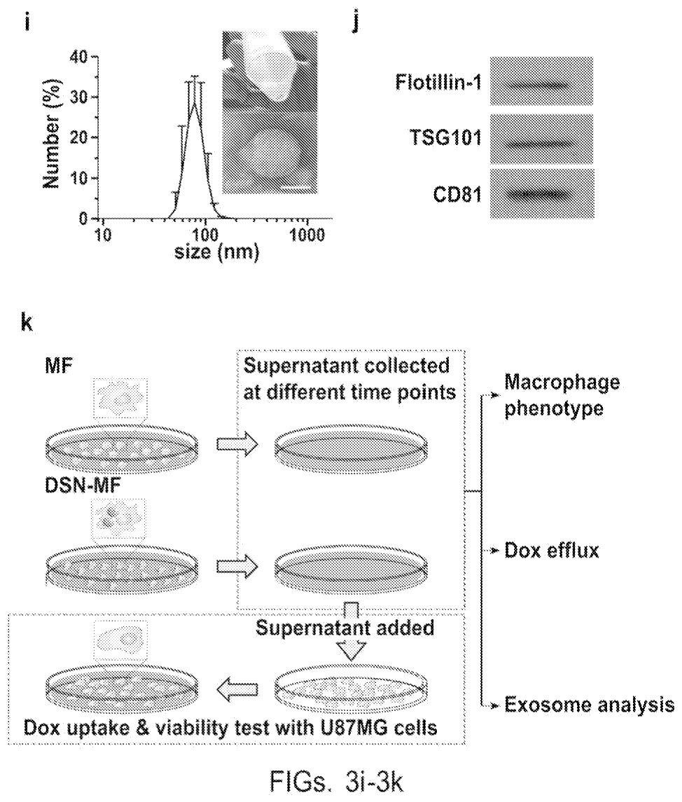

The impact of DSN-52 loading on macrophage phenotype changes was also examined Specifically, the amounts of cytokines, including IL-1β, IL-6, IL-12, TNF-α, and IL-10, that were secreted from DSN-MF were analyzed. Except for IL-1β, which showed comparable secretion relative to the control, other pro-inflammatory markers, including IL-6, IL-12 and TNF-α, all showed significantly elevated secretion (FIG. 3a through 3ₑ). In particular, the IL-6 level was drastically increased from 9.1 pg mL⁻¹ in untreated macrophages to 484.2 pg mL⁻¹ in DSN-MF at 24 h (FIG. 3b). On the contrary, the level of IL-10, an anti-inflammation marker, was reduced from 8.1 pg mL⁻¹ in the control to 4.8 pg mL⁻¹ DSN-MF at 24 h (p<0.001; FIG. 3c). Accompanied with it, the IL-12/IL-10 ratio was increased from 2.2 in MF to 8.0 in DSN-MF (FIG. 3f). These results indicate that RAW264.7 cells after DSN-52 loading were polarized toward the pro-inflammation M1 phenotype.^{[29,30]}

Dox efflux was then analyzed. A time-dependent increase of Dox content in the supernatant of DSN-MF was observed, which released over 50% of the loaded Dox within 48 h (FIG. 3g; Table 4). Supernatants from different time points were added to the incubation media of U87MG cells cultured in separate plates (FIG. 3a). For the 48^{-h} conditioned medium, incubation with U87MG led to extensive cell uptake of Dox (FIG. 12b), which eventually led to cell death (FIG. 3h). Notably, conditioned medium taken at 12 h caused little U87MG cell viability drop (FIG. 3h), which is attributed to the stalled drug release of the nanocapsules.

TABLE 4
Dox release from DSN-MF.

| # | Dox_{load} | Doxₑₓₒ | Doxₛᵤₚ | % Dox | % Dox |
|---|---|---|---|---|---|
| cells | (μg) | (μg) | (μg) | release | (exosome) |
| 5.03 ± | 80.03 ± | 7.51 ± | 37.93 ± | 56.8% | 16.5% |
| 0.13 × | 2.00 | 0.47 | 1.07 | | |
| 10⁶ | | | | | |

Interestingly, Dox was not released entirely in the form of free molecules. When analyzing DSN-MF conditioned medium, it was found that many nanoparticles with a size of 50-150 nm in the supernatant (negative staining TEM; FIG. 3i). Through a series of centrifugation, these nanoparticles were enriched (FIG. 3i). Further Western blotting analysis found high contents of Flotilin-1, TSG101, and CD81 in these nanoparticles (FIG. 3j), suggesting that these were exosomes secreted by macrophages.^{[31]} Spectroscopy analysis revealed that a 16.5% of the released Dox was entrapped within the secreted exosomes (FIGS. 3i and 3j; FIG. 12c; Table 4). Considering possible exosome loss during differential centrifugation, the actual percentage of Dox released in exosomes could be even higher. Unlike artificial liposomes or micelles, exosomes present on their surface adhesion proteins, integrins, and tetraspanins, which may facilitate cancer cell uptake.^{[32,33]} It is envisioned that during therapy, DSN-MF produces Dox-laden exosomes in situ inside tumors, further improving the selectivity and efficiency of the delivery approach.

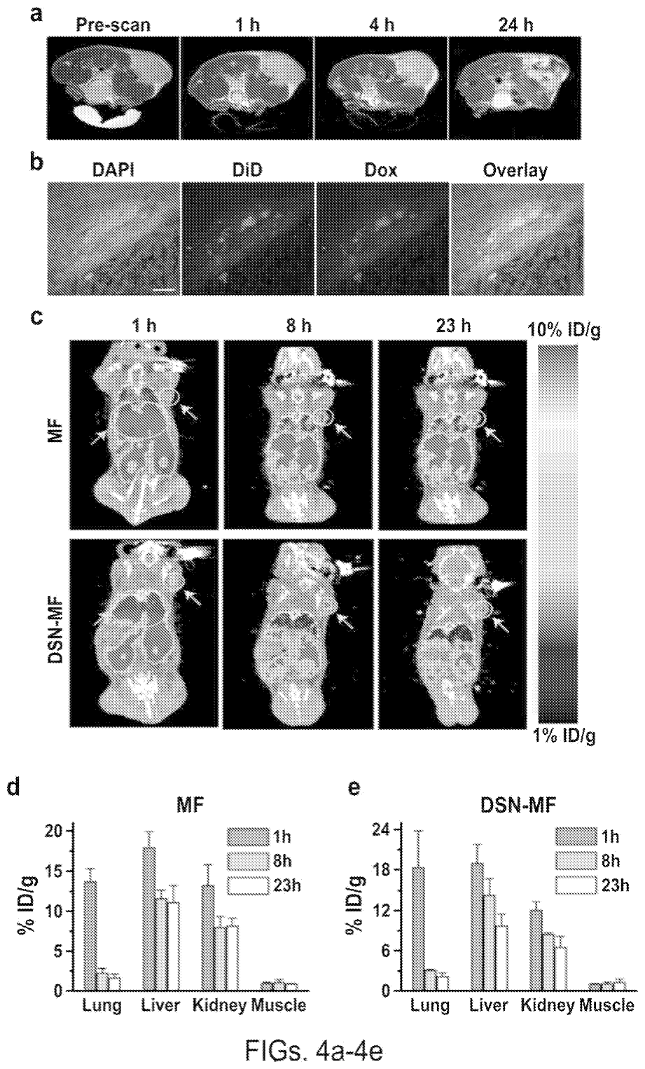

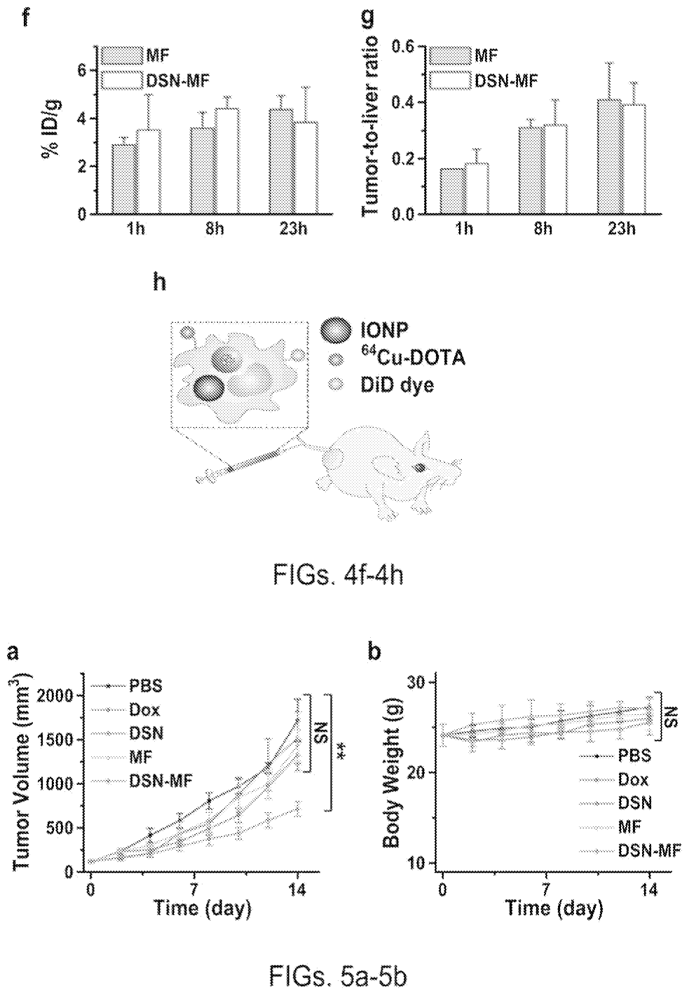

In vivo bio-distribution studies. Tumor tropic properties of DSN-MF were studied in U87MG tumor bearing nude mice. To keep track of the cells, 50 nm iron oxide nanoparticles (IONPs)^{[34,35]} were co-loaded into DSN-MF and i.v. injected 2×10⁶ of the cells into each animal (n=3). T₂-weighted magnetic resonance imaging (MRI) found minimal signal changes in tumors at 4 h, but extensive hypointensities at 24 h (FIG. 4a). To verify cell migration, the cell membrane was labeled with DiD, and tumor samples were examined by histopathology at 24 h post i.v. injection. Positive Prussian blue staining (FIG. 13) was observed within tumors, along with signals from Dox and DiD dye (FIG. 4b), confirming that macrophages as a vehicle can deliver Dox to tumors.

For quantitative analysis, DSN-MF and untreated RAW264.7 cells (MF) were labeled with ⁶⁴Cu-pyruvaldehyde-bis(N4-methylthiosemicarbazone, ⁶⁴Cu-PTSM) and cell migration was monitored by positron emission tomography (PET).^{[36]} It was found that the majority of macrophages were initially accumulated in the lung (FIG. 4c). This was attributed to the pulmonary first-pass effect, which is commonly seen with i.v. injected cells.^{[37,38]} Specifically, the lung uptake at 1 h was 18.30 and 13.64% ID g⁻¹, respectively, for DSN-MF and MF (FIG. 4d and FIG. 4e). Between 1 and 8 h, there was a significant decrease of radioactivity in the lung. Meantime, tumor accumulation was significantly increased (FIG. 4f), suggesting chemotactic migration of macrophages to tumors. For DSN-MF, the decay-adjusted tumor-to-liver ratio (TLR) was increased from 0.18 at 1 h, to 0.32 and 0.39, respectively, at 8 and 23 h (FIG. 4g). These values were not significantly different from the MF group (p>0.05). In general, DSN-MF showed comparable pharmacokinetics to untreated macrophages (FIGS. 4c through 4g), suggesting negligible impact of DSN loading on tumor migration.

Therapy studies. The therapy study was also conducted in U87MG subcutaneous tumor models. Therapy was started when the tumors reached to a size of ˜100 mm³. The animals were randomly sorted to receive the following treatments (n=5) via intravenous injection on Day 0: i) PBS; ii) free Dox (3 mg Dox kg⁻¹); iii) DSN-52 only (3 mg Dox kg⁻¹); iv) untreated RAW264.7 cells (MF, ˜4×10⁶ cells per mouse); v) DSN-MF (3 mg Dox kg⁻¹, ˜4×10⁶ cells per mouse). Only one dose was given to the animals.

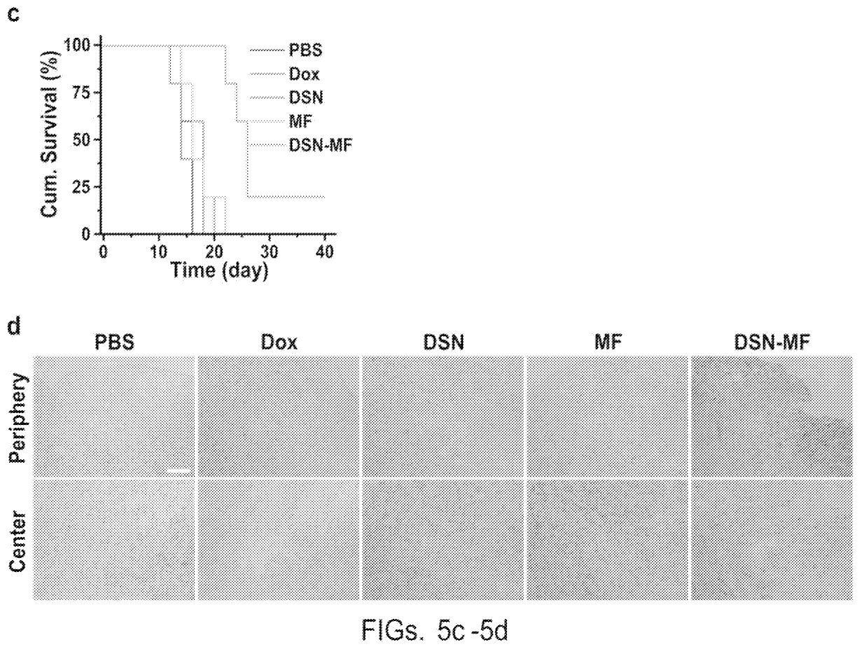

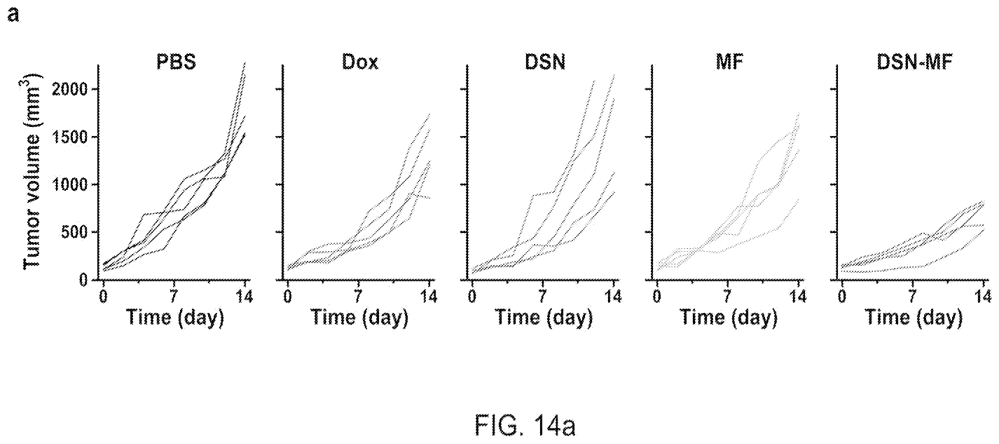

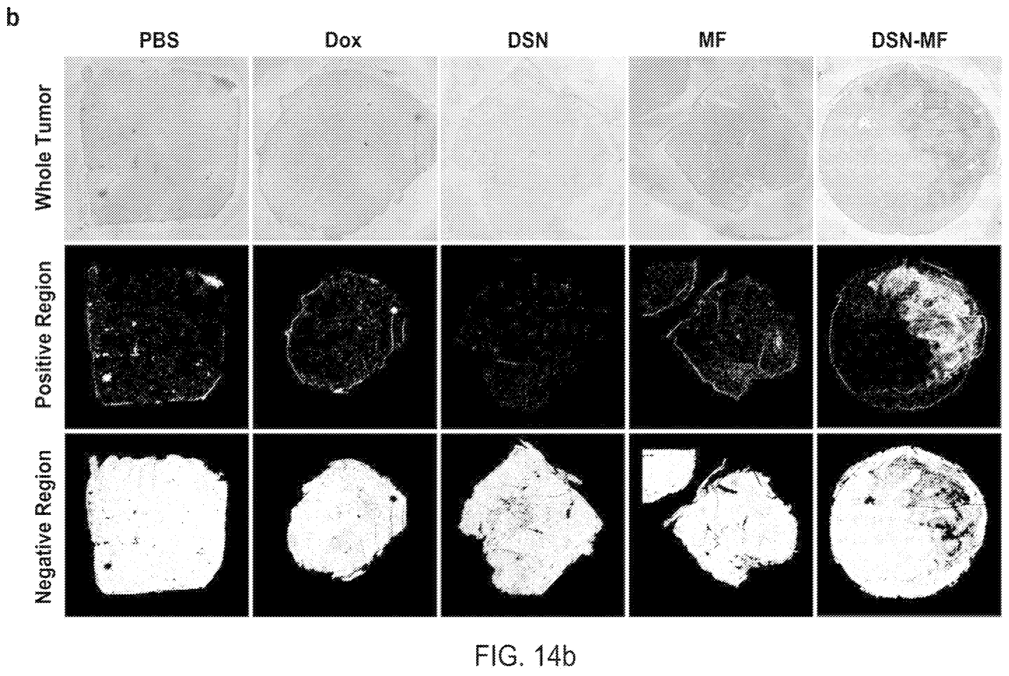

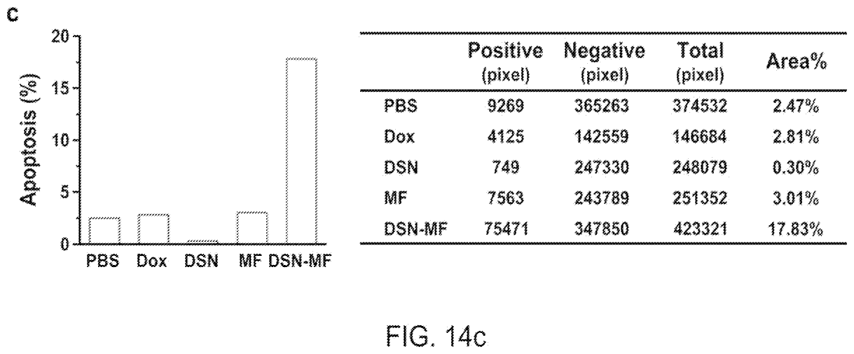

For the Dox, DSN-52, and MF groups, marginal tumor suppression was observed. On Day 14, the average tumor volumes were 1328.6, 1528.47, 1442.27 mm³, respectively, for the three control groups. Relative to the PBS group, the tumor growth inhibition (TGI) rates were 24.84%, 12.35%, and 17.73%, but the changes were insignificant (p=0.17, 0.80, and 0.32, respectively; FIGS. 5a and 14a). There was also no benefit in survival. The median survival was 16, 14, and 14 days, respectively, for the Dox, DSN-52, and MF groups, compared to that of 14 days for the PBS control (FIG. 5c). As a comparison, the DSN-MF group showed an impressive TGI rate of 62.66% on Day 14, and the treatment significantly extended the animal median survival to 26 days. No body weight loss was observed during the whole treatment process (FIG. 5b).

In separate studies, mice were euthanized 24 h after treatment, and performed in situ apoptosis staining (Abcam) on the tumor tissues (FIG. 5d and FIG. 14b). DSN-MF treatment led to extensive cell apoptosis. The positive staining was found at both the peripheral and central tumor areas and occupied 17.83% area of the whole tissue region, which was significantly higher than the controls (FIG. 14c). This is again attributed to the capacity of macrophages to pass biological barriers and migrate to inflamed sites. As a comparison, only sporadic positive staining was observed in the control groups (FIG. 5d).

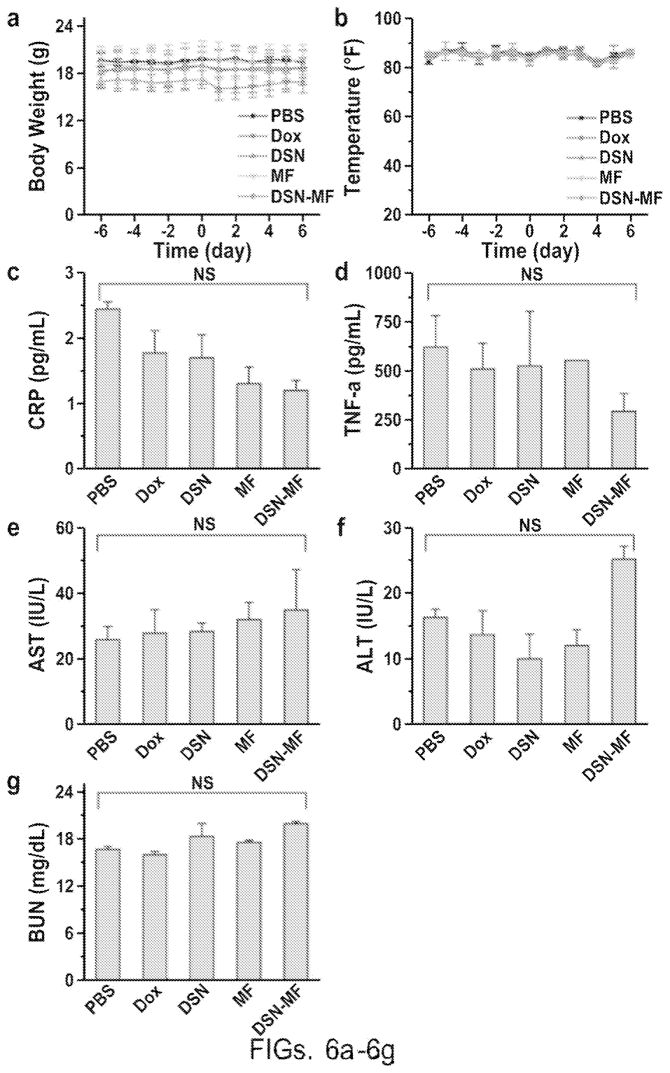

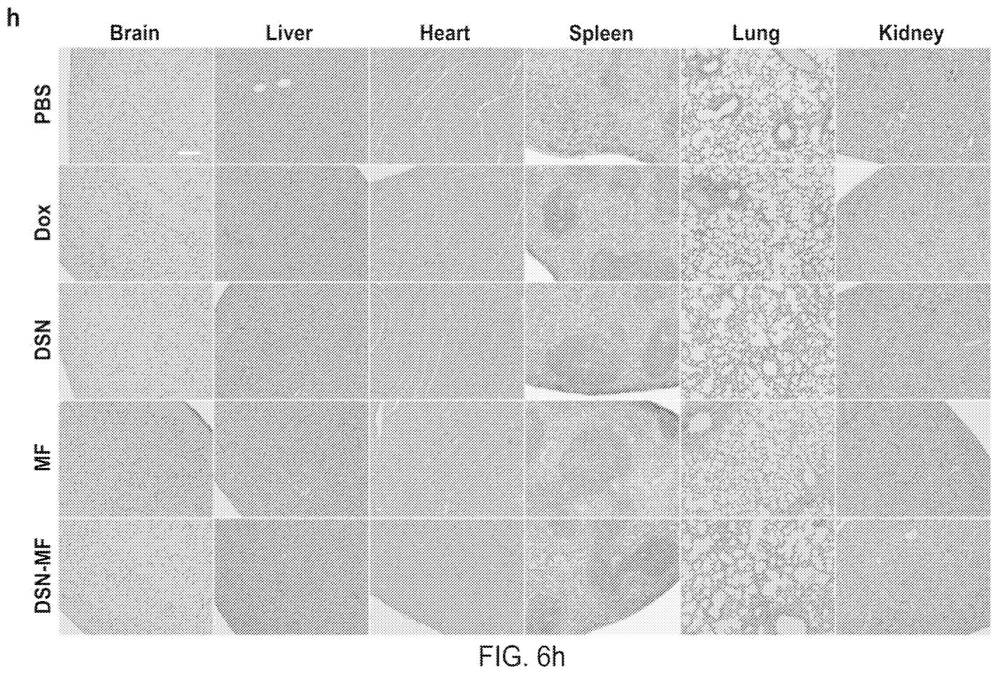

Toxicity studies. It also investigated whether DSN-MF induces systematic toxicity in normal balb/c mice (n=3). In all Dox related groups (Dox, DSN-52, and DSN-MF), animals showed a small degree of body weight loss on Day 2, but the loss was recovered after 3-5 days (FIG. 6a). Meanwhile, there was no detectable change in rectal temperature for all the treatment groups throughout the study (FIG. 6b). After 7 days, the animals were euthanized and major organ tissues were examined by H&E staining. A minor elevation of leukocyte infiltration was observed in the alveolar areas in the DSN-MF group, which is likely attributed to the accumulation of exogenous macrophages.^{[38]} No pathological changes were observed in all other organs (FIG. 6h). These include no detection of cardiotoxicity, which is commonly associated with doxorubicin-based treatments. One concern was that too many activated macrophages may cause increased hemophagocytosis, but there was no evidence of this in the spleen (FIG. 6h).

Blood samples taken from the treated animals were also examined. For the DSN-MF group, complete blood count (CBC) analysis found that all indices were in the normal ranges. As a comparison, the Dox group showed abnormalities including elevated white blood cell and red blood cell counts, high hemoglobin and mean corpuscular hemoglobin levels, low mean corpuscular volumes, low mature neutrophil counts, and elevated immature neutrophil counts (Table 5). These impacts come from hematotoxicity of Dox,^{[39]} which were prevented by selective delivery via macrophages. In addition, protein markers related to inflammation (CRP and TNF-α in FIG. 6c and FIG. 6d),^{[40]} liver function (AST and ALT in FIG. 6e and FIG. 6f), and kidney function (BUN in FIG. 6g) were analyzed. Compared to the control, the ALT level was increased in the DSN-MF group (FIG. 6f), though it was still within the normal range.^{[41]} All the other markers are also within the normal ranges.^{[41]} Overall, the histopathology and blood tests confirmed that DSN-MF induced little systematic toxicity to animals.

TABLE 5
Complete Blood Count Report.

| | | | | | | REF. |
|---|---|---|---|---|---|---|
| | PBS | Dox | DSN | MF | DSN-MF | RANGE |
| WBC (×10³ μL⁻¹) | 5.62 | 13.49 | 6.31 | 3.77 | 8.52 | 6-15 |
| RBC (×10⁶ μL⁻¹) | 9.26 | 13.9 | 9.34 | 8.81 | 8.39 | 7-11 |
| HGB (g dL⁻¹) | 14.0 | 44.0 | 14.1 | 13.7 | 13.1 | 1.2-16.6 |
| HCT (%) | 44.7 | 48.9 | 44.4 | 43.4 | 41.4 | 39-49 |
| MCV (fl) | 48.3 | 15.4 | 47.6 | 49.2 | 49.4 | 41-49 |
| MCH (pg) | 15.1 | 31.6 | 15.1 | 15.5 | 15.6 | 15-18 |
| MCHC (g dL⁻¹) | 31.3 | 62 | 31.8 | 31.6 | 31.7 | 30-38 |
| Segs (%) | 10 | 0 | 13 | 13 | 13 | 10-40 |
| Bands (%) | 0 | 12 | 0 | 0 | 0 | No data |
| Lymphs (%) | 84 | 82 | 79 | 80 | 78 | 55-95 |
| Monos (%) | 6 | 6 | 8 | 7 | 9 | 1-4 |

Inflammation has long been associated with tumor promotion and progression.^{[42,43]} The fact that macrophages or monocytes can respond to chemotactic cues and migrate to inflammation sites has made macrophages a potentially attractive drug delivery vehicle.^{[22,24]} It is envisioned that macrophages can carry therapeutics to tumors, including metastatic sites and tumor central areas, in a highly selective manner. The main challenge of the approach is that it is difficult to load sufficient amounts of drugs onto macrophages. The current study provides a solution. The drug-nanocapsule minimally releases therapeutics in the first 6-12 h of cell entry, permitting one to hijack macrophages as an efficient vehicle to enrich drugs in tumors without killing them pre-maturely. In this context, the phagocytic property of macrophages becomes an advantage, allowing for a very high drug loading (e.g., 16.6 pg cell⁻¹) not possible with the conventional “backpack” approach. DSN-MF i.v. injected are first trapped in the lung but afterward gradually migrate to tumors, with a tumor migration rate comparable to untreated macrophages.^{[44]} While many have attempted to load drugs onto live cells for adoptive cell transfer (ACT)^{[2,7,45,46]} (including neutral stem cells and T cells), the more successful examples are seen with using the approach to improve carrier cell survival and functions.^{[12-14,47]} Due to limited drug loading, exploiting ACT to systematically deliver therapeutic drugs has been a challenge. Here, it is shown that DSN-MF can be injected at a clinically relevant chemotherapeutic dose (3 mg kg⁻¹), which again is due to the high drug loading the nanocapsule approach permits. Post-mortem analysis found extensive cell death in tumors, including the central mass (FIG. 5d and FIGS. 14b through 14c), confirming the benefits of macrophages-based tumor tropism.

For nanoparticle-based drug delivery, drug accumulation in a tumor and its distribution within it rely almost entirely on passive diffusion. Many nanoparticles after extravasation stay in the tumor peripheral region, never reaching the avascular tumor center. Despite a compromised lymphatic system, these nanoparticles are over time drained into the lymphatic system and cleared from the site. Weissleder et al. observed that in many tumors, nanoparticles are first taken up by local macrophages which serve as a depot for continuous drug release.^{[48]} The group also showed that elevating numbers of macrophages in tumors, for instance by external irradiation, provide strongholds for nanoparticles in tumors, leading to improved drug retention and enhanced therapeutic outcomes.^{[49]} In the disclosed strategy, drugs were loaded into macrophages ex vivo but similar stronghold effects should have contributed to the treatment.

While the current study is focused on Dox, it is anticipated that the platform can be easily extended other therapeutics. Given the high loading capacity of macrophages, it is even possible to use the strategy to deliver a multitude of therapeutics to tumors for combination therapy. Moreover, applications are not limited to cancer therapy. Many other diseases, such as tuberculosis, atherosclerosis, and stroke, are also associated with acute or chronic inflammation. Macrophage-based drug delivery may also hold advantages in the treatment of these diseases. The approach may also be used with macrophages derived from autologous monocytes, which is more clinically relevant. It is also possible to load nanocapsules into other cell types such as T cells, neural stem cells, and dendritic cells for drug delivery.

### REFERENCES

[1] Q. Wang, H. Cheng, H. Peng, H. Zhou, P. Y. Li, R. Langer, Adv. Drug Deliv. Rev. 2015, 91, 125.
[2] Y. Su, Z. Xie, G. B. Kim, C. Dong, J. Yang, ACS Biomater. Sci. Eng. 2015, 1, 201.
[3] A. C. Anselmo, S. Mitragotri, J. Control. Release 2014, 190, 531.
[4] L. Yi, H. Xiao, M. Xu, X. Ye, J. Hu, F. Li, M. Li, C. Luo, S. Yu, X. Bian, H. Feng, J. Neuroimmunol. 2011, 232, 75.
[5] A. Singh, M. Talekar, A. Raikar, M. Amiji, J. Control. Release 2014, 190, 515.
[6] S. Li, S. Feng, L. Ding, Y. Liu, Q. Zhu, Z. Qian, Y. Gu, Int. J. Nanomedicine 2016, 11, 4107.
[7] M. Ayer, H. A. Klok, J. Control. Release 2017, 259, 92.
[8] H. Cheng, C. J. Kastrup, R. Ramanathan, D. J. Siegwart, M. Ma, S. R. Bogatyrev, Q. Xu, K. A. Whitehead, R. Langer, D. G. Anderson, ACS Nano 2010, 4, 625.
[9] L. Li, Y. Guan, H. Liu, N. Hao, T. Liu, X. Meng, C. Fu, Y. Li, Q. Qu, Y. Zhang, S. Ji, L. Chen, D. Chen, F. Tang, ACS Nano 2011, 5, 7462.
[10] R. Mooney, Y. Weng, E. Garcia, S. Bhojane, L. Smith-Powell, S. U. Kim, A. J. Annala, K. S. Aboody, J. M. Berlin, J. Control. Release 2014, 191, 82.
[11] R. Mooney, Y. Weng, R. Tirughana-Sambandan, V. Valenzuela, S. Aramburo, E. Garcia, Z. Li, M. Gutova, A. J. Annala, J. M. Berlin, K. S. Aboody, Futur. Oncol. 2014, 10, 401.
[12] M. J. Mitchell, E. C. Wayne, K. Rana, C. B. Schaffer, M. R. King, Proc. IEEE Annu. Northeast Bioeng. Conf. NEBEC 2014, 2014-Decem, 1.
[13] E. C. Wayne, S. Chandrasekaran, M. J. Mitchell, M. F. Chan, R. E. Lee, C. B. Schaffer, M. R. King, J. Control. Release 2016, 223, 215.
[14] S. Chandrasekaran, M. F. Chan, J. Li, M. R. King, Biomaterials 2016, 77, 66.
[15] A. C. Anselmo, J. B. Gilbert, S. Kumar, V. Gupta, R. E. Cohen, M. F. Rubner, S. Mitragotri, J. Control. Release 2015, 199, 29.
[16] W. Tang, Z. Zhen, M. Wang, H. Wang, Y.-J. Chuang, W. Zhang, G. D. Wang, T. Todd, T. Cowger, H. Chen, L. Liu, Z. Li, J. Xie, Adv. Funct. Mater. 2016, 26, 1757.
[17] B. Huang, W. D. Abraham, Y. Zheng, S. C. Bustamante Lopez, S. S. Luo, D. J. Irvine, Sci. Transl. Med. 2015, 7, 291ra94.
[18] M. T. Stephan, J. J. Moon, S. H. Um, A. Bershteyn, D. J. Irvine, Nat. Med. 2010, 16, 1035.
[19] A. Parodi, N. Quattrocchi, A. L. van de Ven, C. Chiappini, M. Evangelopoulos, J. O. Martinez, B. S. Brown, S. Z. Khaled, I. K. Yazdi, M. V. Enzo, L. Isenhart, M. Ferrari, E. Tasciotti, Nat. Nanotechnol. 2013, 8, 61.
[20] R. Molinaro, C. Corbo, J. O. Martinez, F. Taraballi, M. Evangelopoulos, S. Minardi, I. K. Yazdi, P. Zhao, E. De Rosa, M. B. Sherman, A. De Vita, N. E. Toledano Furman, X. Wang, A. Parodi, E. Tasciotti, Nat. Mater. 2016, 15, 1037.
[21] J. Choi, H. Y. Kim, E. J. Ju, J. Jung, J. Park, H. K. Chung, J. S. Lee, J. S. Lee, H. J. Park, S. Y. Song, S. Y. Jeong, E. K. Choi, Biomaterials 2012, 33, 4195.
[22] L. Pang, J. Qin, L. Han, W. Zhao, J. Liang, Z. Xie, P. Yang, J. Wang, Oncotarget 2016, 7, 37081.
[23] W. C. Huang, W. H. Chiang, Y. H. Cheng, W. C. Lin, C. F. Yu, C. Y. Yen, C. K. Yeh, C. S. Chern, C. S. Chiang, H. C. Chiu, Biomaterials 2015, 71, 71.
[24] J. Xie, J. Wang, G. Niu, J. Huang, K. Chen, X. Li, X. Chen, Chem. Commun. (Camb). 2010, 46, 433.
[25] E. Scarpa, J. L. Bailey, A. A. Janeczek, P. S. Stumpf, A. H. Johnston, R. O. C. Oreffo, Y. L. Woo, Y. C. Cheong, N. D. Evans, T. A. Newman, Sci. Rep. 2016, 6, 1.
[26] M. Vandenbranden, G. De Gand, R. Brasseur, F. Defrise-Quertain, J. M. Ruysschaert, Biosci. Rep. 1985, 5, 477.
[27] S. Zhang, Z. Chu, C. Yin, C. Zhang, G. Lin, Q. Li, J. Am. Chem. Soc. 2013, 135, 5709.
[28] J. E. DiCiccio, B. E. Steinberg, J. Gen. Physiol. 2011, 137, 385.
[29] D. M. Mosser, J. P. Edwards, Nat. Rev. Immunol. 2008, 8, 958.
[30] A. Mantovani, S. Sozzani, M. Locati, P. Allavena, A. Sica, Trends Immunol. 2002, 23, 549.
[31] C. Soekmadji, C. C. Nelson, Biomed Res. Int 2015, 2015, 1.
[32] X. Zhang, X. Yuan, H. Shi, L. Wu, H. Qian, W. Xu, J. Hematol. Oncol. 2015, 8, 1.
[33] A. S. Azmi, B. Bao, F. H. Sarkar, and F. H. S. Asfar S Azmil, Bin Bao, Cancer Metastasis Rev. 2014, 32, 1.
[34] Q. Quan, J. Xie, H. Gao, M. Yang, F. Zhang, G. Liu, X. Lin, A. Wang, H. S. Eden, S. Lee, G. Zhang, X. Chen, Mol. Pharm. 2011, 8, 1669.
[35] H. Chen, W. Zhang, G. Zhu, J. Xie, X. Chen, Nat. Rev. Mater. 2017, 2, 17024.
[36] N. Adonai, N. Adonai, K. N. Nguyen, J. Walsh, M. Iyer, T. Toyokuni, M. E. Phelps, T. McCarthy, D. W. McCarthy, S. S. Gambhir, Proc. Natl. Acad. Sci. 2002, 99, 3030.
[37] H. Dou, C. J. Destache, J. R. Morehead, R. L. Mosley, M. D. Boska, J. Kingsley, S. Gorantla, L. Poluektova, J. a. Nelson, M. Chaubal, J. Werling, J. Kipp, B. E. Rabinow, H. E. Gendelman, Blood 2006, 108, 2827.
[38] U. M. Fischer, M. T. Harting, F. Jimenez, W. O. Monzon-Posadas, H. Xue, S. I. Savitz, G. A. Laine, C. S. Cox, Stem Cells Dev. 2009, 18, 683.
[39] D. A. Eppstein, C. G. Kurahara, N. A. Bruno, T. G. Terrell, Cancer Res. 1989, 49, 3955.
[40] MOUSE C-REACTIVE PROTEIN (CRP) ELISA Life Diagnostics, Inc. (lifediagnostics.com)
[41] S. Chen, G. Li, K. Zhu, P. Sun, R. Wang, X. Zhao, Oncol. Lett. 2014, 7, 250.
[42] L. M. Coussens, Z. Werb, Nature 2002, 420, 860.
[43] H. F. Dvorak, Cancer Immunol. Res. 2015, 3, 1.
[44] H. Dou, C. J. Destache, J. R. Morehead, R. L. Mosley, M. D. Boska, J. Kingsley, S. Gorantla, L. Poluektova, J. a. Nelson, M. Chaubal, J. Werling, J. Kipp, B. E. Rabinow, H. E. Gendelman, Blood 2006, 108, 2827.
[45] Z. Xie, Y. Su, G. B. Kim, E. Selvi, C. Ma, V. Aragon-Sanabria, J.-T. Hsieh, C. Dong, J. Yang, Small 2017, 13, 1603121.
[46] E. V. Batrakova, H. E. Gendelman, A. V. Kabanov, Expert Opin Drug Deliv 2011, 8, 415.
[47] S. Lee, E. Choi, M.-J. Cha, K.-C. Hwang, Oxid. Med. Cell. Longev. 2015, 2015, 1.
[48] J. Kim, J. E. Lee, J. Lee, J. H. Yu, B. C. Kim, K. An, Y. Hwang, C.-H. Shin, J.-G. Park, J. Kim, T. Hyeon, J. Am. Chem. Soc. 2006, 128, 688.
[49] M. A. Miller, R. Chandra, M. F. Cuccarese, C. Pfirschke, C. Engblom, S. Stapleton, U. Adhikary, R. H. Kohler, J. F. Mohan, M. J. Pittet, R. Weissleder, Sci. Transl. Med. 2017, 9, eaal0225.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A nanoparticle comprising:
a therapeutic or diagnostic agent positioned within an orthosilicate inner matrix, and
a silica outer shell encapsulating the orthosilicate inner matrix.

2. A method of preparing a nanoparticle comprising:
combining a therapeutic or diagnostic agent and an orthosilicate to form an orthosilicate inner matrix, and
forming a silica outer shell over the orthosilicate inner matrix.

3. A method of treating a disease in a subject comprising administering to the subject a biological carrier cell comprising the nanoparticle of claim 1.

4. A method of delivering a therapeutic or diagnostic agent to a target cell comprising contacting the target cell with a biological carrier cell comprising the nanoparticle of claim 1.

5. A method of loading a therapeutic or diagnostic agent into a biological carrier cell comprising contacting the biological carrier cell with the nanoparticle of claim 1.

6. The nanoparticle of claim 1, wherein the therapeutic or diagnostic agent has a molecular weight of 10,000 g/mol or less.

7. (canceled)

8. (canceled)

9. (canceled)

10. (canceled)

11. (canceled)

12. The nanoparticle of claim 1, wherein two or more therapeutic or diagnostic agents are positioned within the orthosilicate inner matrix.

13. The nanoparticle of claim 1, wherein the orthosilicate inner matrix further comprises additional components which are not diagnostic or therapeutic agents.

14. The nanoparticle of claim 13, wherein the additional components modulate the integrity or continuity of the orthosilicate inner matrix.

15. The nanoparticle of claim 1, wherein the therapeutic or diagnostic agent is an inorganic or biochemical compound.

16. The nanoparticle of claim 1, wherein therapeutic or diagnostic agent is toxic to a biological carrier cell or causes side-effects when administered systematically.

17. The nanoparticle of claim 1, wherein a therapeutically effective dose of the therapeutic agent cannot be loaded into or attached to a biological carrier cell for subsequent administration to a subject.

18. The nanoparticle of claim 1, wherein the therapeutic agent can is an anti-cancer chemotherapeutic, an antimicrobial or antibiotic agent, or an anti-inflammatory agent.

19. The nanoparticle of claim 1, wherein the orthosilicate inner matrix increase susceptibility of the matrix to hydrolysis.

20. The nanoparticle of claim 1, wherein the matrix comprises SiO₂.

21. (canceled)

22. (canceled)

23. (canceled)

24. (canceled)

25. The nanoparticle of claim 1, wherein the orthosilicate inner matrix comprises a silica-containing matrix which can degrade at acidic pH.

26. (canceled)

27. The nanoparticle of claim 1, wherein the orthosilicate inner matrix comprises an orthosilicate attached to one or more organic groups.

28. The nanoparticle of claim 27, wherein the organic group is an alkyl group.

29. The nanoparticle of claim 1, wherein the orthosilicate inner matrix comprises tetramethyl orthosilicate (TMOS), tetraethyl orthosilicate (TEOS), or tetrabutyl orthosilicate (TBOS).

30-55. (canceled)
